SEQUENCE LISTING (25 sequences)

SEQ ID NO: 1 - RNA, 23 nt, synthetic construct
  source 1..23: mol_type = other RNA
taaatggggacccaaaaagaagg

SEQ ID NO: 2 - RNA, 23 nt, synthetic construct
  source 1..23: mol_type = other RNA
accctattgaaaaatgtgcttgg

SEQ ID NO: 3 - RNA, 23 nt, synthetic construct
  source 1..23: mol_type = other RNA
aataaattttgccaaagaaaagg

SEQ ID NO: 4 - RNA, 23 nt, synthetic construct
  source 1..23: mol_type = other RNA
ataaaatgttatgtttaggaagg

SEQ ID NO: 5 - RNA, 25 nt, synthetic construct
  source 1..25: mol_type = other RNA
ctaacttctctgtccctttcaatcc

SEQ ID NO: 6 - RNA, 21 nt, synthetic construct
  source 1..21: mol_type = other RNA
cgctttcttggtcggcttgat

SEQ ID NO: 7 - RNA, 23 nt, synthetic construct
  source 1..23: mol_type = other RNA
gctccatgtgtgtttgattcgac

SEQ ID NO: 8 - DNA, 21 bp, synthetic construct
  source 1..21: mol_type = other DNA
cagcttcaataacgggaattt

SEQ ID NO: 9 - DNA, 20 bp, synthetic construct
  source 1..20: mol_type = other DNA
gttgtaaggtgcagatgagt

SEQ ID NO: 10 - DNA, 19 bp, synthetic construct
  source 1..19: mol_type = other DNA
aacgtggccaaaatgatgc

SEQ ID NO: 11 - DNA, 20 bp, synthetic construct
  source 1..20: mol_type = other DNA
aaccgcttggtcgactatcg

SEQ ID NO: 12 - DNA, 27 bp, synthetic construct
  source 1..27: mol_type = other DNA
gagccttacaacgctactctgtctgtc

SEQ ID NO: 13 - DNA, 27 bp, synthetic construct
  source 1..27: mol_type = other DNA
acaccagacatagtagcagaaatcaag

SEQ ID NO: 14 - DNA, 24 bp, synthetic construct
  source 1..24: mol_type = other DNA
gaaaccaccaccgaagctgttgac

SEQ ID NO: 15 - DNA, 25 bp, synthetic construct
  source 1..25: mol_type = other DNA
gtagcgtcgtcgtaggtccattcac

SEQ ID NO: 16 - RNA, 31 nt, synthetic construct
  source 1..31: mol_type = other RNA
tatataaatggggacccaaaaagaaggtttt

SEQ ID NO: 17 - RNA, 32 nt, synthetic construct
  source 1..32: mol_type = other RNA
tatataaatggggacccaaaaaagaaggtttt

SEQ ID NO: 18 - RNA, 32 nt, synthetic construct
  source 1..32: mol_type = other RNA
tatataaatggggacccaaaatagaaggtttt

SEQ ID NO: 19 - DNA, 22187 bp, synthetic construct
  source 1..22187: mol_type = other DNA
gttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgactctagctagaggatcgatccgaaccccagagtcccgctcagaagaactcgtcaagaaggcgatagaaggcgatgcgctgcgaatcgggagcggcgataccgtaaagcacgaggaagcggtcagcccattcgccgccaagttcttcagcaatatcacgggtagccaacgctatgtcctgatagcggtccgccacacccagccggccacagtcgatgaatccagaaaagcggccattttccaccatgatattcggcaagcaggcatcgccatgtgtcacgacgagatcctcgccgtcgggcatgcgcgccttgagcctggcgaacagttcggctggcgcgagcccctgatgttcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctggagttcattcagggcaccggacaggtcggtcttgacaaaaagaaccgggcgcccctgcgctgacagccgaaacacggcggcatcagagcagccgattgtctgttgtgcccagtcatagccgaatagcctctccacccaagcggccggagaacctgcgtgcaatccatcttgttcaatccccatggtcgatcgacagatctgcgaaagctcgagagagatagatttgtagagagagactggtgatttcagcgtgtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaattcgagctcggtacccggggatcggcgcgccggaggtcgagctgcaggtcaacggatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaacacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacaggaatgaacatggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacgcctatccctatgacgtgcccgattatgccagcctgggcagcggctcccccaagaaaaaacgcaaggtggaagatcctaagaaaaagcggaaagtggaagatgctccaaagaagaagagaaaggtcgacggcattggtagtgggagcaacggcagcagcggatcccttaagcttgccaccatgcgtacgggcaagaatgagcaggaactgctggaactggacaaatgggcaagcctggggagcggctccggaaagaacgagcaggaactgctggaactggacaagtgggcttctctgggctccgggtccgggaaaaacgaacaggagctgctggagctggataagtgggccagcctgggctccgggtccgggaagaatgaacaggaactgctggaactggataaatgggcatctctgggcagcgggtccggaaagaatgaacaggagctgctggaactggacaagtgggcatccctggggagcgggtccggaaagaatgaacaggaactgctggaactggataagtgggcatccctgggaagcggctccggaaaaaacgagcaggagctgctggagctggacaagtgggcctctctgggctccggctccgggaagaatgaacaggagctgctggaactggataagtgggcatctctggggtccggaagcggtaaaaacgagcaggaactgctggaactggacaagtgggcaagcctggggtctggctccggcaaaaacgaacaggaactgctggaactggacaaatgggccagcctgggctccgggtccgggaaaaatgagcaggaactgctggaactggataaatgggcgagcctgggcagcgggtccggaaaaaacgaacaggagctgctggagctggacaaatgggcgagtctgggctccggaagcggcaagaatgaacaggaactgctggagctggacaaatgggccagcctgggcagcggctccggaaagaatgagcaggaactgctggagctggacaaatgggcatctctgggctccggctccgggaaaaacgagcaggaactgctggagctggacaagtgggcttccctgggcagcggctccggaaaaaatgaacaggaactgctggaactggacaagtgggccagcctggggtccgggtctgggaagaacgagcaggaactgctggagctggacaagtgggcatctctggggtctggctctggcaagaacgagcaggaactgctggaactggacaagtgggcgagcctgggcagcgggtctggcaagaatgaacaggagctgctggaactggacaaatgggccagcctggggtctgggtctggaaagaatgagcaggaactgctggagctggacaagtgggcatctctgggctctgggtctggcaagaacgagcaggagctgctggagctggataagtgggcatccctggggtccgggtccggcaagaatgaacaggagctgctggagctggataaatgggcatccctgggctctgggtctgggaagaatgagcaggagctgctggaactggataagtgggcgagcctgggcagcgggtctggcaagaatgagcaggaactgctggagctggacaagtgggcttctctgcaccggtcagcagtggaggacagcacgtaacccgggcttatatgaagatgaagatgaaatatttggtgtgtcaaataaaaagcttgtgtgcttaagtttgtgtttttttcttggcttgttgtgttatgaatttgtggctttttctaatattaaatgaatgtaagatcacattataatgaataaacaaatgtttctataatccattgtgaatgttttgttggatctcttctgcagcatataactactgtatgtgctatggtatggactatggaatatgattaaagataagcgctgagctcggacccatggcttcgttgaacaacggaaactcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgtaaatggggacccaaaaagagtttaagagctatgctggaaacagcatagcaagtttaaataaggctagtccgttatcaacttgaaaaagtggcaccgagtcggtgctttttttcccggggcgcgccctatggagttctagaatgtcgcggaacaaattttaaaactaaatcctaaatttttctaattttgttgccaatagtggatatgtgggccgtatagaaggaatctattgaaggcccaaacccatactgacgagcccaaaggttcgttttgcgttttatgtttcggttcgatgccaacgccacattctgagctaggcaaaaaacaaacgtgtctttgaatagactcctctcgttaacacatgcagcggctgcatggtgacgccattaacacgtggcctacaattgcatgatgtctccattgacacgtgacttctcgtctcctttcttaatatatctaacaaacactcctacctcttccaaaatatatacacatctttttgatcaatctctcattcaaaatctcattctctctagtaaacaagaacaaaaaaatggcggatacagctagaggaacccatcacgatatcatcggcagagatcagtacccgatgatgggccgagatcgtgaccagtaccagatgtccggacgaggatctgactactccaagtctaggcagattgctaaagctgcaactgctgtcacagctggtggttccctccttgttctctccagccttacccttgttggaactgtcatagctttgactgttgcaacacctctgctcgttatcttcagcccaatccttgtcccggctctcatcacagttgcactcctcatcaccggttttctttcctctggagggtttggcattgccgctataaccgttttctcttggatttacaagtaagcacacatttatcatcttacttcataattttgtgcaatatgtgcatgcatgtgttgagccagtagctttggatcaatttttttggtcgaataacaaatgtaacaataagaaattgcaaattctagggaacatttggttaactaaatacgaaatttgacctagctagcttgaatgtgtctgtgtatatcatctatataggtaaaatgcttggtatgatacctattgattgtgaataggtacgcaacgggagagcacccacagggatcagacaagttggacagtgcaaggatgaagttgggaagcaaagctcaggatctgaaagacagagctcagtactacggacagcaacatactggtggggaacatgaccgtgaccgtactcgtggtggccagcacactactatgagcgagctgattaaggagaacatgcacatgaagctgtacatggagggcaccgtgaacaaccaccacttcaagtgcacatccgagggcgaaggcaagccctacgagggcacccagaccatgagaatcaaggtggtcgagggcggccctctccccttcgccttcgacatcctggctaccagcttcatgtacggcagcagaaccttcatcaaccacacccagggcatccccgacttctttaagcagtccttccctgagggcttcacatgggagagagtcaccacatacgaagatgggggcgtgctgaccgctacccaggacaccagcctccaggacggctgcctcatctacaacgtcaagatcagaggggtgaacttcccatccaacggccctgtgatgcagaagaaaacactcggctgggaggccaacaccgagatgctgtaccccgctgacggcggcctggaaggcagaagcgacatggccctgaagctcgtgggcgggggccacctgatctgcaacttcaagaccacatacagatccaagaaacccgctaagaacctcaagatgcccggcgtctactatgtggaccacagactggaaagaatcaaggaggccgacaaagaaacctacgtcgagcagcacgaggtggctgtggccagatactgcgacctccctagcaaactggggcacaagtgagcttaccccactgatgtcatcgtcatagtccaataactccaatgtcggggagttagtttatgaggaataaagtgtttagaatttgatcagggggagataataaaagccgagtttgaatctttttgttataagtaatgtttatgtgtgtttctatatgttgtcaaatggtaccatgttttttttcctctctttttgtaacttgcaagtgttgtgttgtactttatttggcttctttgtaagttggtaacggtggtctatatatggaaaaggtcttgttttgttaaacttatgttagttaactggattcgtctttaaccacaaaaagttttcaataagctacaaatttagacacgcaagccgatgcagtcattagtacatatatttattgcaagtgattacatggcaacccaaacttcaaaaacagtaggttgctccatttagtcgctgttagagctctcccggcgcgccggaggtcgagctgcaggtcaacggatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaacacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacaggaatgccaaagaagaagaggaaagtcatggaggtgcagctggtggaatctgggggcggactggtgcagcccgggggatctctgcggctgtcctgcgccgcctctggctccatctctagcgtggatgtgatgtcctggtacaggcaggcccctggcaagcagagggaactggtggccttcattactgataggggaagaaccaattacaaagtgagcgtgaaaggccgcttcactatcagccgggataattctaagaatatggtgtatctgcagatgaacagcctgaagccagaagacactgccgactatctgtgcagggctgagtcccggacttcctggtccagcccatctcctctggatgtgtgggggcgcggcactcaggtgactgtgtcctccttggatccaggtggaggtggaagcggtagcaaaggagaagaacttttcactggagttgtcccaattcttgttgaattagatggtgatgttaatgggcacaaattttctgtccgtggagagggtgaaggtgatgctacaaacggaaaactcacccttaaatttatttgcactactggaaaactacctgttccgtggccaacacttgtcactactctgacctatggtgttcaatgcttttcccgttatccggatcacatgaaacggcatgactttttcaagagtgccatgcccgaaggttatgtacaggaacgcactatatctttcaaagatgacgggacctacaagacgcgtgctgaagtcaagtttgaaggtgatacccttgttaatcgtatcgagttaaagggtattgattttaaagaagatggaaacattcttggacacaaactcgagtacaactttaactcacacaatgtatacatcacggcagacaaacaaaagaatggaatcaaagctaacttcaaaattcgccacaacgttgaagatggttccgttcaactagcagaccattatcaacaaaatactccaattggcgatggccctgtccttttaccagacaaccattacctgtcgacacaatctgtcctttcgaaagatcccaacgaaaagcgtgaccacatggtccttcttgagtttgtaactgctgctgggattacacatggcatggatgagctctacaaaggtagtggcggtggtggttcaggaggcggcggaagcttagatccaggtggaggtggaggtagcaccgtgatgtgggaacaggatgcagccccctttgcaggcgcagccgatgactttccagcttttaacgaggaggaattagcgtggctgatggagctgctccctcaaagcggatctgtgggggggaccattggaggtggtagtggtggaggtcggaccgaagagtacaagcttatcctgaacggtaaaaccctgaaaggtgaaaccaccaccgaagctgttgacgctgctaccgcggaaaaagttttcaaacagtacgctaacgacaacggtgttgacggtgaatggacctacgacgacgctaccaaaaccttcacggtaaccgaataagcttgttcgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtcgctgagctcagtgtggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacggctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggcctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagcccaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggaaacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggccctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagtt

SEQ ID NO: 20 - DNA, 15449 bp, synthetic construct
  source 1..15449: mol_type = other DNA
ttaggcggaaaaatcgccttttttctcttttatatcagtcacttacatgtgtgaccggttcccaatgtacggctttgggttcccaatgtacgggttccggttcccaatgtacggctttgggttcccaatgtacgtgctatccacaggaaagaggccttttcgacctttttcccctgctagggcaatttgccctagcatctgctccgtacattaggaaccggcggatgcttcgccctcgatcaggttgcggtagcgcatgactaggatcgggccagcctgccccgcctcctccttcaaatcgtactccggcaggtcatttgacccgatcagcttgcgcacggtgaaacagaacttcttgaactctccggcgctgccactgcgttcgtagatcgtcttgaacaaccatctggcttctgccttgcctgcggcgcggcgtgccaggcggtagagaaaacggccgatgccgggatcgatcaaaaagtaatcggggtgaaccgtcagcacgtccgggttcttgccttctgtgatctcgcggtacatccaatcagctagctcgatctcgatgtactccggccgcccggtttcgctctttacgatcttgtagcggctaatcaaggcttcaccctcggataccgtcaccaggcggccgttcttggccttcttcgtacgctgcatggcaacgtgcgtggtgtttaaccgaatgcaggtttctaccaggtcgtctttctgctttccgccatcggctcgccggcagaacttgagtacgtccgcaacgtgtggacggaacacgcggccgggcttgtctcccttcccttcccggtatcggttcatggattcggttagatgggaaaccgccatcagtaccaggtcgtaatcccacacactggccatgccggccggccctgcggaaacctctacgtgcccgtctggaagctcgtagcggatcacctcgccagctcgtcggtcacgcttcgacagacggaaaacggccacgtccatgatgctgcgactatcgcgggtgcccacgtcatagagcatcggaacgaaaaaatctggttgctcgtcgcccttgggcggcttcctaatcgacggcgcaccggctgccggcggttgccgggattctttgcggattcgatcagcggccgcttgccacgattcaccggggcgtgcttctgcctcgatgcgttgccgctgggcggcctgcgcggccttcaacttctccaccaggtcatcacccagcgccgcgccgatttgtaccgggccggatggtttgcgaccgtcacgccgattcctcgggcttgggggttccagtgccattgcagggccggcagacaacccagccgcttacgcctggccaaccgcccgttcctccacacatggggcattccacggcgtcggtgcctggttgttcttgattttccatgccgcctcctttagccgctaaaattcatctactcatttattcatttgctcatttactctggtagctgcgcgatgtattcagatagcagctcggtaatggtcttgccttggcgtaccgcgtacatcttcagcttggtgtgatcctccgccggcaactgaaagttgacccgcttcatggctggcgtgtctgccaggctggccaacgttgcagccttgctgctgcgtgcgctcggacggccggcacttagcgtgtttgtgcttttgctcattttctctttacctcattaactcaaatgagttttgatttaatttcagcggccagcgcctggacctcgcgggcagcgtcgccctcgggttctgattcaagaacggttgtgccggcggcggcagtgcctgggtagctcacgcgctgcgtgatacgggactcaagaatgggcagctcgtacccggccagcgcctcggcaacctcaccgccgatgcgcgtgcctttgatcgcccgcgacacgacaaaggccgcttgtagccttccatccgtgacctcaatgcgctgcttaaccagctccaccaggtcggcggtggcccatatgtcgtaagggcttggctgcaccggaatcagcacgaagtcggctgccttgatcgcggacacagccaagtccgccgcctggggcgctccgtcgatcactacgaagtcgcgccggccgatggccttcacgtcgcggtcaatcgtcgggcggtcgatgccgacaacggttagcggttgatcttcccgcacggccgcccaatcgcgggcactgccctggggatcggaatcgactaacagaacatcggccccggcgagttgcagggcgcgggctagatgggttgcgatggtcgtcttgcctgacccgcctttctggttaagtacagcgataaccttcatgcgttccccttgcgtatttgtttatttactcatcgcatcatatacgcagcgaccgcatgacgcaagctgttttactcaaatacacatcacctttttagacggcggcgctcggtttcttcagcggccaagctggccggccaggccgccagcttggcatcagacaaaccggccaggatttcatgcagccgcacggttgagccgtgcgcgggcggctcgaacacgtacccggccgcgatcatctccgcctcgatctcttcggtaatgaaaaacggttcgtcctggccgtcctggtgcggtttcatgcttgttcctcttggcgttcattctcggcggccgccagggcgtcggcctcggtcaatgcgtcctcacggaaggcaccgcgccgcctggcctcggtgggcgtcacttcctcgctgcgctcaagtgcgcggtacagggtcgagcgatgcacgccaagcagtgcagccgcctctttcacggtgcggccttcctggtcgatcagctcgcgggcgtgcgcgatctgtgccggggtgagggtagggcgggggccaaacttcacgcctcgggccttggcggcctcgcgcccgctccgggtgcggtcgatgattagggaacgctcgaactcggcaatgccggcgaacacggtcaacaccatgcggccggccggcgtggtggtgtcggcccacggctctgccaggctacgcaggcccgcgccggcctcctggatgcgctcggcaatgtccagtaggtcgcgggtgctgcgggccaggcggtctagcctggtcactgtcacaacgtcgccagggcgtaggtggtcaagcatcctggccagctccgggcggtcgcgcctggtgccggtgatcttctcggaaaacagcttggtgcagccggccgcgtgcagttcggcccgttggttggtcaagtcctggtcgtcggtgctgacgcgggcatagcccagcaggccagcggcggcgctcttgttcatggcgtaatgtctccggttctagtcgcaagtattctactttatgcgactaaaacacgcgacaagaaaacgccaggaaaagggcagggcggcagcctgtcgcgtaacttaggacttgtgcgacatgtcgttttcagaagacggctgcactgaacgtcagaagccgactgcactatagcagcggaggggttggatcaaagtactttgatcccgaggggaaccctgtggttggcatgcacatacaaatggacgaacggataaaccttttcacgcccttttaaatatccgttattctaataaacgctcttttctcttaggtttacccgccaatatatcctgtcaaacactgatagtttttcgtacgaccctcggtaccgatcggcgcgccggagctcaacttttgattcgctatttgcagtgcacctgtggcgttcatcacatcttttgtgacactgtttgcactggtcattgctattacaaaggaccttcctgatgttgaaggagatcgaaagtaagtaactgcacgcataaccattttctttccgctctttggctcaatccatttgacagtcaaagacaatgtttaaccagctccgtttgatatattgtctttatgtgtttgttcaagcatgtttagttaatcatgcctttgattgatcttgaataggttccaaatatcaaccctggcaacaaaacttggagtgagaaacattgcattcctcggttctggacttctgctagtaaattatgtttcagccatatcactagctttctacatgcctcaggtgaattcatctatttccgtcttaactatttcggttaattaaagcacgaacaccattactgcatgtagaagcttgataaactatcgccaccaatttatttttgttgcgatattgttactttcctcagtatgcagctttgaaaagaccaaccctcttatcctttaacaatgaacaggtttttagaggtagcttgatgattcctgcacatgtgatcttggcttcaggcttaattttccaggtaaagcattatgagatactcttatatctcttacatacttttgagataatgcacaagaacttcataactatatgctttagtttctgcatttgacactgccaaattcattaatctctaatatctttgttgttgatctttggtagacatgggtactagaaaaagcaaactacaccaaggtaaaatacttttgtacaaacataaactcgttatcacggaacatcaatggagtgtatatctaacggagtgtagaaacatttgattattgcaggaagctatctcaggatattatcggtttatatggaatctcttctacgcagagtatctgttattccccttcctctagctttcaatttcatggtgaggatatgcagttttctttgtatatcattcttcttcttctttgtagcttggagtcaaaatcggttccttcatgtacatacatcaaggatatgtccttctgaatttttatatcttgcaataaaaatgcttgtaccaattgaaacaccagctttttgagttctatgatcactgacttggttctaaccaaaaaaaaaaaaatgtttaatttacatatctaaaagtaggtttagggaaacctaaacagtaaaatatttgtatattattcgaatttcactcatcataaaaacttaaattgcaccataaaattttgttttactattaatgatgtaatttgtgtaacttaagataaaaataatattccgtaagttaaccggctaaaaccacgtataaaccagggaacctgttaaaccggttctttactggataaagaaatgaaagcccatgtagacagctccattagagcccaaaccctaaatttctcatctatataaaaggagtgacattagggtttttgttcgtcctcttaaagcttctcgttttctctgccgtctctctcattcgcgcgacgcaaacgatcttcaggtgatcttctttctccaaatcctctctcataactctgatttcgtacttgtgtatttgagctcacgctctgtttctctcaccacagcaatggataagaagtactctatcggactcgatatcggaactaactctgtgggatgggctgtgatcaccgatgagtacaaggtgccatctaagaagttcaaggttctcggaaacaccgataggcactctatcaagaaaaaccttatcggtgctctcctcttcgattctggtgaaactgctgaggctaccagactcaagagaaccgctagaagaaggtacaccagaagaaagaacaggatctgctacctccaagagatcttctctaacgagatggctaaagtggatgattcattcttccacaggctcgaagagtcattcctcgtggaagaagataagaagcacgagaggcaccctatcttcggaaacatcgttgatgaggtggcataccacgagaagtaccctactatctaccacctcagaaagaagctcgttgattctactgataaggctgatctcaggctcatctacctcgctctcgctcacatgatcaagttcagaggacacttcctcatcgagggtgatctcaaccctgataactctgatgtggataagttgttcatccagctcgtgcagacctacaaccagcttttcgaagagaaccctatcaacgcttcaggtgtggatgctaaggctatcctctctgctaggctctctaagtcaagaaggcttgagaacctcattgctcagctccctggtgagaagaagaacggacttttcggaaacttgatcgctctctctctcggactcacccctaacttcaagtctaacttcgatctcgctgaggatgcaaagctccagctctcaaaggatacctacgatgatgatctcgataacctcctcgctcagatcggagatcagtacgctgatttgttcctcgctgctaagaacctctctgatgctatcctcctcagtgatatcctcagagtgaacaccgagatcaccaaggctccactctcagcttctatgatcaagagatacgatgagcaccaccaggatctcacacttctcaaggctcttgttagacagcagctcccagagaagtacaaagagattttcttcgatcagtctaagaacggatacgctggttacatcgatggtggtgcatctcaagaagagttctacaagttcatcaagcctatcctcgagaagatggatggaaccgaggaactcctcgtgaagctcaatagagaggatcttctcagaaagcagaggaccttcgataacggatctatccctcatcagatccacctcggagagttgcacgctatccttagaaggcaagaggatttctacccattcctcaaggataacagggaaaagattgagaagattctcaccttcagaatcccttactacgtgggacctctcgctagaggaaactcaagattcgcttggatgaccagaaagtctgaggaaaccatcaccccttggaacttcgaagaggtggtggataagggtgctagtgctcagtctttcatcgagaggatgaccaacttcgataagaaccttccaaacgagaaggtgctccctaagcactctttgctctacgagtacttcaccgtgtacaacgagttgaccaaggttaagtacgtgaccgagggaatgaggaagcctgcttttttgtcaggtgagcaaaagaaggctatcgttgatctcttgttcaagaccaacagaaaggtgaccgtgaagcagctcaaagaggattacttcaagaaaatcgagtgcttcgattcagttgagatttctggtgttgaggataggttcaacgcatctctcggaacctaccacgatctcctcaagatcattaaggataaggatttcttggataacgaggaaaacgaggatatcttggaggatatcgttcttaccctcaccctctttgaagatagagagatgattgaagaaaggctcaagacctacgctcatctcttcgatgataaggtgatgaagcagttgaagagaagaagatacactggttggggaaggctctcaagaaagctcattaacggaatcagggataagcagtctggaaagacaatccttgatttcctcaagtctgatggattcgctaacagaaacttcatgcagctcatccacgatgattctctcacctttaaagaggatatccagaaggctcaggtttcaggacagggtgatagtctccatgagcatatcgctaacctcgctggatctcctgcaatcaagaagggaatcctccagactgtgaaggttgtggatgagttggtgaaggtgatgggaaggcataagcctgagaacatcgtgatcgaaatggctagagagaaccagaccactcagaagggacagaagaactctagggaaaggatgaagaggatcgaggaaggtatcaaagagcttggatctcagatcctcaaagagcaccctgttgagaacactcagctccagaatgagaagctctacctctactacctccagaacggaagggatatgtatgtggatcaagagttggatatcaacaggctctctgattacgatgttgatcatatcgtgccacagtcattcttgaaggatgattctatcgataacaaggtgctcaccaggtctgataagaacaggggtaagagtgataacgtgccaagtgaagaggttgtgaagaaaatgaagaactattggaggcagctcctcaacgctaagctcatcactcagagaaagttcgataacttgactaaggctgagaggggaggactctctgaattggataaggcaggattcatcaagaggcagcttgtggaaaccaggcagatcactaagcacgttgcacagatcctcgattctaggatgaacaccaagtacgatgagaacgataagttgatcagggaagtgaaggttatcaccctcaagtcaaagctcgtgtctgatttcagaaaggatttccaattctacaaggtgagggaaatcaacaactaccaccacgctcacgatgcttaccttaacgctgttgttggaaccgctctcatcaagaagtatcctaagctcgagtcagagttcgtgtacggtgattacaaggtgtacgatgtgaggaagatgatcgctaagtctgagcaagagatcggaaaggctaccgctaagtatttcttctactctaacatcatgaatttcttcaagaccgagattaccctcgctaacggtgagatcagaaagaggccactcatcgagacaaacggtgaaacaggtgagatcgtgtgggataagggaagggatttcgctaccgttagaaaggtgctctctatgccacaggtgaacatcgttaagaaaaccgaggtgcagaccggtggattctctaaagagtctatcctccctaagaggaactctgataagctcattgctaggaagaaggattgggaccctaagaaatacggtggtttcgattctcctaccgtggcttactctgttctcgttgtggctaaggttgagaagggaaagagtaagaagctcaagtctgttaaggaacttctcggaatcactatcatggaaaggtcatctttcgagaagaacccaatcgatttcctcgaggctaagggatacaaagaggttaagaaggatctcatcatcaagctcccaaagtactcactcttcgaactcgagaacggtagaaagaggatgctcgcttctgctggtgagcttcaaaagggaaacgagcttgctctcccatctaagtacgttaactttctttacctcgcttctcactacgagaagttgaagggatctccagaagataacgagcagaagcaacttttcgttgagcagcacaagcactacttggatgagatcatcgagcagatctctgagttctctaaaagggtgatcctcgctgatgcaaacctcgataaggtgttgtctgcttacaacaagcacagagataagcctatcagggaacaggcagagaacatcatccatctcttcacccttaccaacctcggtgctcctgctgctttcaagtacttcgatacaaccatcgataggaagagatacacctctaccaaagaagtgctcgatgctaccctcatccatcagtctatcactggactctacgagactaggatcgatctctcacagctcggtggtgattcaagggctgatcctaagaagaagaggaaggtttgagcttatatgaagatgaagatgaaatatttggtgtgtcaaataaaaagcttgtgtgcttaagtttgtgtttttttcttggcttgttgtgttatgaatttgtggctttttctaatattaaatgaatgtaagatcacattataatgaataaacaaatgtttctataatccattgtgaatgttttgttggatctcttctgcagcatataactactgtatgtgctatggtatggactatggaatatgattaaagataagcgctgagctcggacccatggcttcgttgaacaacggaaactcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgtaaatggggacccaaaaagagtttaagagctatgctggaaacagcatagcaagtttaaataaggctagtccgttatcaacttgaaaaagtggcaccgagtcggtgctttttttcccggggcgcgccctatggagttctagaatgtcgcggaacaaattttaaaactaaatcctaaatttttctaattttgttgccaatagtggatatgtgggccgtatagaaggaatctattgaaggcccaaacccatactgacgagcccaaaggttcgttttgcgttttatgtttcggttcgatgccaacgccacattctgagctaggcaaaaaacaaacgtgtctttgaatagactcctctcgttaacacatgcagcggctgcatggtgacgccattaacacgtggcctacaattgcatgatgtctccattgacacgtgacttctcgtctcctttcttaatatatctaacaaacactcctacctcttccaaaatatatacacatctttttgatcaatctctcattcaaaatctcattctctctagtaaacaagaacaaaaaaatggcggatacagctagaggaacccatcacgatatcatcggcagagatcagtacccgatgatgggccgagatcgtgaccagtaccagatgtccggacgaggatctgactactccaagtctaggcagattgctaaagctgcaactgctgtcacagctggtggttccctccttgttctctccagccttacccttgttggaactgtcatagctttgactgttgcaacacctctgctcgttatcttcagcccaatccttgtcccggctctcatcacagttgcactcctcatcaccggttttctttcctctggagggtttggcattgccgctataaccgttttctcttggatttacaagtaagcacacatttatcatcttacttcataattttgtgcaatatgtgcatgcatgtgttgagccagtagctttggatcaatttttttggtcgaataacaaatgtaacaataagaaattgcaaattctagggaacatttggttaactaaatacgaaatttgacctagctagcttgaatgtgtctgtgtatatcatctatataggtaaaatgcttggtatgatacctattgattgtgaataggtacgcaacgggagagcacccacagggatcagacaagttggacagtgcaaggatgaagttgggaagcaaagctcaggatctgaaagacagagctcagtactacggacagcaacatactggtggggaacatgaccgtgaccgtactcgtggtggccagcacactactatgagcgagctgattaaggagaacatgcacatgaagctgtacatggagggcaccgtgaacaaccaccacttcaagtgcacatccgagggcgaaggcaagccctacgagggcacccagaccatgagaatcaaggtggtcgagggcggccctctccccttcgccttcgacatcctggctaccagcttcatgtacggcagcagaaccttcatcaaccacacccagggcatccccgacttctttaagcagtccttccctgagggcttcacatgggagagagtcaccacatacgaagatgggggcgtgctgaccgctacccaggacaccagcctccaggacggctgcctcatctacaacgtcaagatcagaggggtgaacttcccatccaacggccctgtgatgcagaagaaaacactcggctgggaggccaacaccgagatgctgtaccccgctgacggcggcctggaaggcagaagcgacatggccctgaagctcgtgggcgggggccacctgatctgcaacttcaagaccacatacagatccaagaaacccgctaagaacctcaagatgcccggcgtctactatgtggaccacagactggaaagaatcaaggaggccgacaaagaaacctacgtcgagcagcacgaggtggctgtggccagatactgcgacctccctagcaaactggggcacaagtgagcttaccccactgatgtcatcgtcatagtccaataactccaatgtcggggagttagtttatgaggaataaagtgtttagaatttgatcagggggagataataaaagccgagtttgaatctttttgttataagtaatgtttatgtgtgtttctatatgttgtcaaatggtaccatgttttttttcctctctttttgtaacttgcaagtgttgtgttgtactttatttggcttctttgtaagttggtaacggtggtctatatatggaaaaggtcttgttttgttaaacttatgttagttaactggattcgtctttaaccacaaaaagttttcaataagctacaaatttagacacgcaagccgatgcagtcattagtacatatatttattgcaagtgattacatggcaacccaaacttcaaaaacagtaggttgctccatttagtcgctgttagagctcagtgtttgatcgccggcggtaccgagtgtacttcaagtcactcaattcggcgttaattcagtacattaaaaacgtccgcaatgtgttattaagttgtctaagcgtcaatttgtttacaccacaatatatcctgccaccagccagccaacagctccccgaccggcagctcggcacaaaatcaccactcgatacaggcagcccatcagtccgggacggcgtcagcgggagagccgttgtaaggcggcagactttgctcatgttaccgatgctattcggaagaacggcaactaagctgccgggtttgaaacacggatgatctcgcggagggtagcatgttgattgtaacgatgacagagcgttgctgcctgtgatcaccgcggtttcaaaatcggctccgtcgatactatgttatacgccaactttgaaaacaactttgaaaaagctgttttctggtatttaaggttttagaatgcaaggaacagtgaattggagttcgtcttgttataattagcttcttggggtatctttaaatactgtagaaaagaggaaggaaataataaatggctaaaatgagaatatcaccggaattgaaaaaactgatcgaaaaataccgctgcgtaaaagatacggaaggaatgtctcctgctaaggtatataagctggtgggagaaaatgaaaacctatatttaaaaatgacggacagccggtataaagggaccacctatgatgtggaacgggaaaaggacatgatgctatggctggaaggaaagctgcctgttccaaaggtcctgcactttgaacggcatgatggctggagcaatctgctcatgagtgaggccgatggcgtcctttgctcggaagagtatgaagatgaacaaagccctgaaaagattatcgagctgtatgcggagtgcatcaggctctttcactccatcgacatatcggattgtccctatacgaatagcttagacagccgcttagccgaattggattacttactgaataacgatctggccgatgtggattgcgaaaactgggaagaagacactccatttaaagatccgcgcgagctgtatgattttttaaagacggaaaagcccgaagaggaacttgtcttttcccacggcgacctgggagacagcaacatctttgtgaaagatggcaaagtaagtggctttattgatcttgggagaagcggcagggcggacaagtggtatgacattgccttctgcgtccggtcgatcagggaggatatcggggaagaacagtatgtcgagctattttttgacttactggggatcaagcctgattgggagaaaataaaatattatattttactggatgaattgttttagtacctagaatgcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtttccgggagctgcatgtgtcagaggttttcaccgtcatcaccgaaacgcgcgaggcagggtgccttgatgtgggcgccggcggtcgagtggcgacggcgcggcttgtccgcgccctggtagattgcctggccgtaggccagccatttttgagcggccagcggccgcgataggccgacgcgaagcggcggggcgtagggagcgcagcgaccgaagggtaggcgctttttgcagctcttcggctgtgcgctggccagacagttatgcacaggccaggcgggttttaagagttttaataagttttaaagagtt

SEQ ID NO: 21 - DNA, 4368 bp, synthetic construct
  source 1..4368: mol_type = other DNA
aatgtcgcggaacaaattttaaaactaaatcctaaatttttctaattttgttgccaatagtggatatgtgggccgtatagaaggaatctattgaaggcccaaacccatactgacgagcccaaaggttcgttttgcgttttatgtttcggttcgatgccaacgccacattctgagctaggcaaaaaacaaacgtgtctttgaatagactcctctcgttaacacatgcagcggctgcatggtgacgccattaacacgtggcctacaattgcatgatgtctccattgacacgtgacttctcgtctcctttcttaatatatctaacaaacactcctacctcttccaaaatatatacacatctttttgatcaatctctcattcaaaatctcattctctctagtaaacaagaacaaaaaaatggcggatacagctagaggaacccatcacgatatcatcggcagagatcagtacccgatgatgggccgagatcgtgaccagtaccagatgtccggacgaggatctgactactccaagtctaggcagattgctaaagctgcaactgctgtcacagctggtggttccctccttgttctctccagccttacccttgttggaactgtcatagctttgactgttgcaacacctctgctcgttatcttcagcccaatccttgtcccggctctcatcacagttgcactcctcatcaccggttttctttcctctggagggtttggcattgccgctataaccgttttctcttggatttacaagtaagcacacatttatcatcttacttcataattttgtgcaatatgtgcatgcatgtgttgagccagtagctttggatcaatttttttggtcgaataacaaatgtaacaataagaaattgcaaattctagggaacatttggttaactaaatacgaaatttgacctagctagcttgaatgtgtctgtgtatatcatctatataggtaaaatgcttggtatgatacctattgattgtgaataggtacgcaacgggagagcacccacagggatcagacaagttggacagtgcaaggatgaagttgggaagcaaagctcaggatctgaaagacagagctcagtactacggacagcaacatactggtggggaacatgaccgtgaccgtactcgtggtggccagcacactactatgagcgagctgattaaggagaacatgcacatgaagctgtacatggagggcaccgtgaacaaccaccacttcaagtgcacatccgagggcgaaggcaagccctacgagggcacccagaccatgagaatcaaggtggtcgagggcggccctctccccttcgccttcgacatcctggctaccagcttcatgtacggcagcagaaccttcatcaaccacacccagggcatccccgacttctttaagcagtccttccctgagggcttcacatgggagagagtcaccacatacgaagatgggggcgtgctgaccgctacccaggacaccagcctccaggacggctgcctcatctacaacgtcaagatcagaggggtgaacttcccatccaacggccctgtgatgcagaagaaaacactcggctgggaggccaacaccgagatgctgtaccccgctgacggcggcctggaaggcagaagcgacatggccctgaagctcgtgggcgggggccacctgatctgcaacttcaagaccacatacagatccaagaaacccgctaagaacctcaagatgcccggcgtctactatgtggaccacagactggaaagaatcaaggaggccgacaaagaaacctacgtcgagcagcacgaggtggctgtggccagatactgcgacctccctagcaaactggggcacaagtgagcttaccccactgatgtcatcgtcatagtccaataactccaatgtcggggagttagtttatgaggaataaagtgtttagaatttgatcagggggagataataaaagccgagtttgaatctttttgttataagtaatgtttatgtgtgtttctatatgttgtcaaatggtaccatgttttttttcctctctttttgtaacttgcaagtgttgtgttgtactttatttggcttctttgtaagttggtaacggtggtctatatatggaaaaggtcttgttttgttaaacttatgttagttaactggattcgtctttaaccacaaaaagttttcaataagctacaaatttagacacgcaagccgatgcagtcattagtacatatatttattgcaagtgattacatggcaacccaaacttcaaaaacagtaggttgctccatttagtcgctgttagagctctcccactagcaggtgatgaaaagccttagggtcaggtggcacttttcggggaaatgtgcgatgaacccctatttgtttatttttctaaatacattcaaatatgtatccgctaatgagacaataaccctgataaatgcttcaattatattgaaaaaggaagagtatgagtatccaacatttccgtgtcgcccttattcccttttttgcggccttctgccttcctgtttttgctcacccagaaacgctggtgaaagtaaaagatgctgaagatcagttgggtgcccgagtgggttacattgaactggatctcaacagcggcaaaatcttagagagttttcgccccgaagaacgcttcccaatgatgagcactttcaaagttctgctatgtggcgcggtgttatcccgtattgatgctgggcaagagcaactcggtcgccgcatacactattctcagaatgacttggttgagtattcaccagtcacagaaaagcatcttacggatggcatgacagtaagagaattatgtagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaactctccgcaaactattaactggcgaactacttactcttgcttcacgccagcaactcattgactggatggaggcggacaaagttgcaggaccacttctgcgctcggcacttccggctggctggtttattgctgataaatctggagccggtgagcgtggctctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagacgaagggaaataaatagtagcccgcctgatgtgcgggcttttttttggtggtggttaccagtgtcgcttagctaccgtaggtcctcttaggcggtccgattagcgacatagtcatcaatgccttgacggcaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctactaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgctcacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccacctgcaacgccggggcgcgccctatggagttctag

SEQ ID NO: 22 - DNA, 9389 bp, synthetic construct
  source 1..9389: mol_type = other DNA
gtcgagctgcaggtcaacggatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaacacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacaggaatgaacatggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacgcctatccctatgacgtgcccgattatgccagcctgggcagcggctcccccaagaaaaaacgcaaggtggaagatcctaagaaaaagcggaaagtggaagatgctccaaagaagaagagaaaggtcgacggcattggtagtgggagcaacggcagcagcggatcccttaagcttgccaccatgcgtacgggcaagaatgagcaggaactgctggaactggacaaatgggcaagcctggggagcggctccggaaagaacgagcaggaactgctggaactggacaagtgggcttctctgggctccgggtccgggaaaaacgaacaggagctgctggagctggataagtgggccagcctgggctccgggtccgggaagaatgaacaggaactgctggaactggataaatgggcatctctgggcagcgggtccggaaagaatgaacaggagctgctggaactggacaagtgggcatccctggggagcgggtccggaaagaatgaacaggaactgctggaactggataagtgggcatccctgggaagcggctccggaaaaaacgagcaggagctgctggagctggacaagtgggcctctctgggctccggctccgggaagaatgaacaggagctgctggaactggataagtgggcatctctggggtccggaagcggtaaaaacgagcaggaactgctggaactggacaagtgggcaagcctggggtctggctccggcaaaaacgaacaggaactgctggaactggacaaatgggccagcctgggctccgggtccgggaaaaatgagcaggaactgctggaactggataaatgggcgagcctgggcagcgggtccggaaaaaacgaacaggagctgctggagctggacaaatgggcgagtctgggctccggaagcggcaagaatgaacaggaactgctggagctggacaaatgggccagcctgggcagcggctccggaaagaatgagcaggaactgctggagctggacaaatgggcatctctgggctccggctccgggaaaaacgagcaggaactgctggagctggacaagtgggcttccctgggcagcggctccggaaaaaatgaacaggaactgctggaactggacaagtgggccagcctggggtccgggtctgggaagaacgagcaggaactgctggagctggacaagtgggcatctctggggtctggctctggcaagaacgagcaggaactgctggaactggacaagtgggcgagcctgggcagcgggtctggcaagaatgaacaggagctgctggaactggacaaatgggccagcctggggtctgggtctggaaagaatgagcaggaactgctggagctggacaagtgggcatctctgggctctgggtctggcaagaacgagcaggagctgctggagctggataagtgggcatccctggggtccgggtccggcaagaatgaacaggagctgctggagctggataaatgggcatccctgggctctgggtctgggaagaatgagcaggagctgctggaactggataagtgggcgagcctgggcagcgggtctggcaagaatgagcaggaactgctggagctggacaagtgggcttctctgcaccggtcagcagtggaggacagcacgtaacccgggcttatatgaagatgaagatgaaatatttggtgtgtcaaataaaaagcttgtgtgcttaagtttgtgtttttttcttggcttgttgtgttatgaatttgtggctttttctaatattaaatgaatgtaagatcacattataatgaataaacaaatgtttctataatccattgtgaatgttttgttggatctcttctgcagcatataactactgtatgtgctatggtatggactatggaatatgattaaagataagcgctgagctcggacttttgcaggtgtgatgaaaagccttagggtcaggtggcacttttcggggaaatgtgcgatgaacccctatttgtttatttttctaaatacattcaaatatgtatccgctaatgagacaataaccctgataaatgcttcaattatattgaaaaaggaagagtatgagtatccaacatttccgtgtcgcccttattcccttttttgcggccttctgccttcctgtttttgctcacccagaaacgctggtgaaagtaaaagatgctgaagatcagttgggtgcccgagtgggttacattgaactggatctcaacagcggcaaaatcttagagagttttcgccccgaagaacgcttcccaatgatgagcactttcaaagttctgctatgtggcgcggtgttatcccgtattgatgctgggcaagagcaactcggtcgccgcatacactattctcagaatgacttggttgagtattcaccagtcacagaaaagcatcttacggatggcatgacagtaagagaattatgtagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaactctccgcaaactattaactggcgaactacttactcttgcttcacgccagcaactcattgactggatggaggcggacaaagttgcaggaccacttctgcgctcggcacttccggctggctggtttattgctgataaatctggagccggtgagcgtggctctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagacgaagggaaataaatagtagcccgcctgatgtgcgggcttttttttggtggtggttaccagtgtcgcttagctaccgtaggtcctcttaggcggtccgattagcgacatagtcatcaatgccttgacggcaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctactaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgctcacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccacctgcttttgatcggcgcgccggag

SEQ ID NO: 23 - DNA, 2587 bp, synthetic construct
  source 1..2587: mol_type = other DNA
cttcgttgaacaacggaaactcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgtaaatggggacccaaaaagagtttaagagctatgctggaaacagcatagcaagtttaaataaggctagtccgttatcaacttgaaaaagtggcaccgagtcggtgctttttttcccggtgacgcaggtgatgaaaagccttagggtcaggtggcacttttcggggaaatgtgcgatgaacccctatttgtttatttttctaaatacattcaaatatgtatccgctaatgagacaataaccctgataaatgcttcaattatattgaaaaaggaagagtatgagtatccaacatttccgtgtcgcccttattcccttttttgcggccttctgccttcctgtttttgctcacccagaaacgctggtgaaagtaaaagatgctgaagatcagttgggtgcccgagtgggttacattgaactggatctcaacagcggcaaaatcttagagagttttcgccccgaagaacgcttcccaatgatgagcactttcaaagttctgctatgtggcgcggtgttatcccgtattgatgctgggcaagagcaactcggtcgccgcatacactattctcagaatgacttggttgagtattcaccagtcacagaaaagcatcttacggatggcatgacagtaagagaattatgtagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaactctccgcaaactattaactggcgaactacttactcttgcttcacgccagcaactcattgactggatggaggcggacaaagttgcaggaccacttctgcgctcggcacttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagacgaagggaaataaatagtagcccgcctgatgtgcgggcttttttttggtggtggttaccagtgtcgcttagctaccgtaggtcctcttaggcggtccgattagcgacatagtcatcaatgccttgacggcaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctactaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgctcacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccacctgccaggggacccatgg

SEQ ID NO: 24 - DNA, 5204 bp, synthetic construct
  source 1..5204: mol_type = other DNA
gtcgagctgcaggtcaacggatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaacacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacaggaatgccaaagaagaagaggaaagtcatggaggtgcagctggtggaatctgggggcggactggtgcagcccgggggatctctgcggctgtcctgcgccgcctctggctccatctctagcgtggatgtgatgtcctggtacaggcaggcccctggcaagcagagggaactggtggccttcattactgataggggaagaaccaattacaaagtgagcgtgaaaggccgcttcactatcagccgggataattctaagaatatggtgtatctgcagatgaacagcctgaagccagaagacactgccgactatctgtgcagggctgagtcccggacttcctggtccagcccatctcctctggatgtgtgggggcgcggcactcaggtgactgtgtcctccttggatccaggtggaggtggaagcggtagcaaaggagaagaacttttcactggagttgtcccaattcttgttgaattagatggtgatgttaatgggcacaaattttctgtccgtggagagggtgaaggtgatgctacaaacggaaaactcacccttaaatttatttgcactactggaaaactacctgttccgtggccaacacttgtcactactctgacctatggtgttcaatgcttttcccgttatccggatcacatgaaacggcatgactttttcaagagtgccatgcccgaaggttatgtacaggaacgcactatatctttcaaagatgacgggacctacaagacgcgtgctgaagtcaagtttgaaggtgatacccttgttaatcgtatcgagttaaagggtattgattttaaagaagatggaaacattcttggacacaaactcgagtacaactttaactcacacaatgtatacatcacggcagacaaacaaaagaatggaatcaaagctaacttcaaaattcgccacaacgttgaagatggttccgttcaactagcagaccattatcaacaaaatactccaattggcgatggccctgtccttttaccagacaaccattacctgtcgacacaatctgtcctttcgaaagatcccaacgaaaagcgtgaccacatggtccttcttgagtttgtaactgctgctgggattacacatggcatggatgagctctacaaaggtagtggcggtggtggttcaggaggcggcggaagcttagatccaggtggaggtggaggtagcaccgtgatgtgggaacaggatgcagccccctttgcaggcgcagccgatgactttccagcttttaacgaggaggaattagcgtggctgatggagctgctccctcaaagcggatctgtgggggggaccattggaggtggtagtggtggaggtcggaccgaagagtacaagcttatcctgaacggtaaaaccctgaaaggtgaaaccaccaccgaagctgttgacgctgctaccgcggaaaaagttttcaaacagtacgctaacgacaacggtgttgacggtgaatggacctacgacgacgctaccaaaaccttcacggtaaccgaataagcttgttcgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtcgctgagctcagtgttttgcaggtgtgatgaaaagccttagggtcaggtggcacttttcggggaaatgtgcgatgaacccctatttgtttatttttctaaatacattcaaatatgtatccgctaatgagacaataaccctgataaatgcttcaattatattgaaaaaggaagagtatgagtatccaacatttccgtgtcgcccttattcccttttttgcggccttctgccttcctgtttttgctcacccagaaacgctggtgaaagtaaaagatgctgaagatcagttgggtgcccgagtgggttacattgaactggatctcaacagcggcaaaatcttagagagttttcgccccgaagaacgcttcccaatgatgagcactttcaaagttctgctatgtggcgcggtgttatcccgtattgatgctgggcaagagcaactcggtcgccgcatacactattctcagaatgacttggttgagtattcaccagtcacagaaaagcatcttacggatggcatgacagtaagagaattatgtagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaactctccgcaaactattaactggcgaactacttactcttgcttcacgccagcaactcattgactggatggaggcggacaaagttgcaggaccacttctgcgctcggcacttccggctggctggtttattgctgataaatctggagccggtgagcgtggctctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagacgaagggaaataaatagtagcccgcctgatgtgcgggcttttttttggtggtggttaccagtgtcgcttagctaccgtaggtcctcttaggcggtccgattagcgacatagtcatcaatgccttgacggcaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctactaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgctcacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccacctgctttttcccggcgcgccggag

SEQ ID NO: 25 - DNA, 8099 bp, synthetic construct
  source 1..8099: mol_type = other DNA
gagctcggacttttgcaggtgtgatgaaaagccttagggtcaggtggcacttttcggggaaatgtgcgatgaacccctatttgtttatttttctaaatacattcaaatatgtatccgctaatgagacaataaccctgataaatgcttcaattatattgaaaaaggaagagtatgagtatccaacatttccgtgtcgcccttattcccttttttgcggccttctgccttcctgtttttgctcacccagaaacgctggtgaaagtaaaagatgctgaagatcagttgggtgcccgagtgggttacattgaactggatctcaacagcggcaaaatcttagagagttttcgccccgaagaacgcttcccaatgatgagcactttcaaagttctgctatgtggcgcggtgttatcccgtattgatgctgggcaagagcaactcggtcgccgcatacactattctcagaatgacttggttgagtattcaccagtcacagaaaagcatcttacggatggcatgacagtaagagaattatgtagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaactctccgcaaactattaactggcgaactacttactcttgcttcacgccagcaactcattgactggatggaggcggacaaagttgcaggaccacttctgcgctcggcacttccggctggctggtttattgctgataaatctggagccggtgagcgtggctctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagacgaagggaaataaatagtagcccgcctgatgtgcgggcttttttttggtggtggttaccagtgtcgcttagctaccgtaggtcctcttaggcggtccgattagcgacatagtcatcaatgccttgacggcaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctactaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgctcacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccacctgcttttgatcggcgcgccggagctcaacttttgattcgctatttgcagtgcacctgtggcgttcatcacatcttttgtgacactgtttgcactggtcattgctattacaaaggaccttcctgatgttgaaggagatcgaaagtaagtaactgcacgcataaccattttctttccgctctttggctcaatccatttgacagtcaaagacaatgtttaaccagctccgtttgatatattgtctttatgtgtttgttcaagcatgtttagttaatcatgcctttgattgatcttgaataggttccaaatatcaaccctggcaacaaaacttggagtgagaaacattgcattcctcggttctggacttctgctagtaaattatgtttcagccatatcactagctttctacatgcctcaggtgaattcatctatttccgtcttaactatttcggttaattaaagcacgaacaccattactgcatgtagaagcttgataaactatcgccaccaatttatttttgttgcgatattgttactttcctcagtatgcagctttgaaaagaccaaccctcttatcctttaacaatgaacaggtttttagaggtagcttgatgattcctgcacatgtgatcttggcttcaggcttaattttccaggtaaagcattatgagatactcttatatctcttacatacttttgagataatgcacaagaacttcataactatatgctttagtttctgcatttgacactgccaaattcattaatctctaatatctttgttgttgatctttggtagacatgggtactagaaaaagcaaactacaccaaggtaaaatacttttgtacaaacataaactcgttatcacggaacatcaatggagtgtatatctaacggagtgtagaaacatttgattattgcaggaagctatctcaggatattatcggtttatatggaatctcttctacgcagagtatctgttattccccttcctctagctttcaatttcatggtgaggatatgcagttttctttgtatatcattcttcttcttctttgtagcttggagtcaaaatcggttccttcatgtacatacatcaaggatatgtccttctgaatttttatatcttgcaataaaaatgcttgtaccaattgaaacaccagctttttgagttctatgatcactgacttggttctaaccaaaaaaaaaaaaatgtttaatttacatatctaaaagtaggtttagggaaacctaaacagtaaaatatttgtatattattcgaatttcactcatcataaaaacttaaattgcaccataaaattttgttttactattaatgatgtaatttgtgtaacttaagataaaaataatattccgtaagttaaccggctaaaaccacgtataaaccagggaacctgttaaaccggttctttactggataaagaaatgaaagcccatgtagacagctccattagagcccaaaccctaaatttctcatctatataaaaggagtgacattagggtttttgttcgtcctcttaaagcttctcgttttctctgccgtctctctcattcgcgcgacgcaaacgatcttcaggtgatcttctttctccaaatcctctctcataactctgatttcgtacttgtgtatttgagctcacgctctgtttctctcaccacagcaatggataagaagtactctatcggactcgatatcggaactaactctgtgggatgggctgtgatcaccgatgagtacaaggtgccatctaagaagttcaaggttctcggaaacaccgataggcactctatcaagaaaaaccttatcggtgctctcctcttcgattctggtgaaactgctgaggctaccagactcaagagaaccgctagaagaaggtacaccagaagaaagaacaggatctgctacctccaagagatcttctctaacgagatggctaaagtggatgattcattcttccacaggctcgaagagtcattcctcgtggaagaagataagaagcacgagaggcaccctatcttcggaaacatcgttgatgaggtggcataccacgagaagtaccctactatctaccacctcagaaagaagctcgttgattctactgataaggctgatctcaggctcatctacctcgctctcgctcacatgatcaagttcagaggacacttcctcatcgagggtgatctcaaccctgataactctgatgtggataagttgttcatccagctcgtgcagacctacaaccagcttttcgaagagaaccctatcaacgcttcaggtgtggatgctaaggctatcctctctgctaggctctctaagtcaagaaggcttgagaacctcattgctcagctccctggtgagaagaagaacggacttttcggaaacttgatcgctctctctctcggactcacccctaacttcaagtctaacttcgatctcgctgaggatgcaaagctccagctctcaaaggatacctacgatgatgatctcgataacctcctcgctcagatcggagatcagtacgctgatttgttcctcgctgctaagaacctctctgatgctatcctcctcagtgatatcctcagagtgaacaccgagatcaccaaggctccactctcagcttctatgatcaagagatacgatgagcaccaccaggatctcacacttctcaaggctcttgttagacagcagctcccagagaagtacaaagagattttcttcgatcagtctaagaacggatacgctggttacatcgatggtggtgcatctcaagaagagttctacaagttcatcaagcctatcctcgagaagatggatggaaccgaggaactcctcgtgaagctcaatagagaggatcttctcagaaagcagaggaccttcgataacggatctatccctcatcagatccacctcggagagttgcacgctatccttagaaggcaagaggatttctacccattcctcaaggataacagggaaaagattgagaagattctcaccttcagaatcccttactacgtgggacctctcgctagaggaaactcaagattcgcttggatgaccagaaagtctgaggaaaccatcaccccttggaacttcgaagaggtggtggataagggtgctagtgctcagtctttcatcgagaggatgaccaacttcgataagaaccttccaaacgagaaggtgctccctaagcactctttgctctacgagtacttcaccgtgtacaacgagttgaccaaggttaagtacgtgaccgagggaatgaggaagcctgcttttttgtcaggtgagcaaaagaaggctatcgttgatctcttgttcaagaccaacagaaaggtgaccgtgaagcagctcaaagaggattacttcaagaaaatcgagtgcttcgattcagttgagatttctggtgttgaggataggttcaacgcatctctcggaacctaccacgatctcctcaagatcattaaggataaggatttcttggataacgaggaaaacgaggatatcttggaggatatcgttcttaccctcaccctctttgaagatagagagatgattgaagaaaggctcaagacctacgctcatctcttcgatgataaggtgatgaagcagttgaagagaagaagatacactggttggggaaggctctcaagaaagctcattaacggaatcagggataagcagtctggaaagacaatccttgatttcctcaagtctgatggattcgctaacagaaacttcatgcagctcatccacgatgattctctcacctttaaagaggatatccagaaggctcaggtttcaggacagggtgatagtctccatgagcatatcgctaacctcgctggatctcctgcaatcaagaagggaatcctccagactgtgaaggttgtggatgagttggtgaaggtgatgggaaggcataagcctgagaacatcgtgatcgaaatggctagagagaaccagaccactcagaagggacagaagaactctagggaaaggatgaagaggatcgaggaaggtatcaaagagcttggatctcagatcctcaaagagcaccctgttgagaacactcagctccagaatgagaagctctacctctactacctccagaacggaagggatatgtatgtggatcaagagttggatatcaacaggctctctgattacgatgttgatcatatcgtgccacagtcattcttgaaggatgattctatcgataacaaggtgctcaccaggtctgataagaacaggggtaagagtgataacgtgccaagtgaagaggttgtgaagaaaatgaagaactattggaggcagctcctcaacgctaagctcatcactcagagaaagttcgataacttgactaaggctgagaggggaggactctctgaattggataaggcaggattcatcaagaggcagcttgtggaaaccaggcagatcactaagcacgttgcacagatcctcgattctaggatgaacaccaagtacgatgagaacgataagttgatcagggaagtgaaggttatcaccctcaagtcaaagctcgtgtctgatttcagaaaggatttccaattctacaaggtgagggaaatcaacaactaccaccacgctcacgatgcttaccttaacgctgttgttggaaccgctctcatcaagaagtatcctaagctcgagtcagagttcgtgtacggtgattacaaggtgtacgatgtgaggaagatgatcgctaagtctgagcaagagatcggaaaggctaccgctaagtatttcttctactctaacatcatgaatttcttcaagaccgagattaccctcgctaacggtgagatcagaaagaggccactcatcgagacaaacggtgaaacaggtgagatcgtgtgggataagggaagggatttcgctaccgttagaaaggtgctctctatgccacaggtgaacatcgttaagaaaaccgaggtgcagaccggtggattctctaaagagtctatcctccctaagaggaactctgataagctcattgctaggaagaaggattgggaccctaagaaatacggtggtttcgattctcctaccgtggcttactctgttctcgttgtggctaaggttgagaagggaaagagtaagaagctcaagtctgttaaggaacttctcggaatcactatcatggaaaggtcatctttcgagaagaacccaatcgatttcctcgaggctaagggatacaaagaggttaagaaggatctcatcatcaagctcccaaagtactcactcttcgaactcgagaacggtagaaagaggatgctcgcttctgctggtgagcttcaaaagggaaacgagcttgctctcccatctaagtacgttaactttctttacctcgcttctcactacgagaagttgaagggatctccagaagataacgagcagaagcaacttttcgttgagcagcacaagcactacttggatgagatcatcgagcagatctctgagttctctaaaagggtgatcctcgctgatgcaaacctcgataaggtgttgtctgcttacaacaagcacagagataagcctatcagggaacaggcagagaacatcatccatctcttcacccttaccaacctcggtgctcctgctgctttcaagtacttcgatacaaccatcgataggaagagatacacctctaccaaagaagtgctcgatgctaccctcatccatcagtctatcactggactctacgagactaggatcgatctctcacagctcggtggtgattcaagggctgatcctaagaagaagaggaaggtttgagcttatatgaagatgaagatgaaatatttggtgtgtcaaataaaaagcttgtgtgcttaagtttgtgtttttttcttggcttgttgtgttatgaatttgtggctttttctaatattaaatgaatgtaagatcacattataatgaataaacaaatgtttctataatccattgtgaatgttttgttggatctcttctgcagcatataactactgtatgtgctatggtatggactatggaatatgattaaagataagcgct

(19) United States
(12) Patent Application Publication
(10) Pub. No.: US 2026/0071229 A1
(43) Pub. Date: Mar. 12, 2026

(54) ENGINEERED GENETIC INCOMPATIBILITY IN PLANTS

(71) Applicant: REGENTS OF THE UNIVERSITY OF MINNESOTA, Minneapolis, MN (US)

(72) Inventors: Michael J. Smanski, Falcon Heights, MN (US); Matthew H. Zinselmeier, Des Moines, IA (US); Juan Armando Casas Mollano, St. Louis, MO (US)

(21) Appl. No.: 19/326,385

(22) Filed: Sep. 11, 2025

Related U.S. Application Data
(60) Provisional application No. 63/693,796, filed on Sep. 12, 2024.

(51) Int. Cl. C12N 15/82; C12N 9/22; C12N 15/11

(52) U.S. Cl. CPC C12N 15/8263; C12N 9/226; C12N 15/111; C12N 15/8201; C07K 2319/71; C12N 2310/20

(57) ABSTRACT

In one aspect, a transgenic plant cell includes a biocontainment system that includes: a coding region whose overexpression alters growth of the cell or impairs cell division; a transcription regulatory region operably linked upstream of the coding region, and including a mutation; and a polynucleotide that encodes a programmable transcription activator engineered to bind to the transcription regulatory region in the absence of the mutation after sexual reproduction, thereby overexpressing the coding region in the absence of the mutation, but does not initiate overexpression of the coding region when the transcription regulatory region includes the mutation.

### CROSS-REFERENCE TO RELATED APPLICATIONS

This application claims the benefit of U.S. Provisional Application Ser. No. 63/693,796, filed on Sep. 12, 2024, the disclosure of which is incorporated by reference herein in its entirety.

### GOVERNMENT FUNDING

This invention was made with government support under HR0011-18-C-0146 awarded by the Defense Advanced Research Projects Agency, and 2018-33522-28747 awarded by the National Institute of Food and Agriculture. The government has certain rights in the invention.

### SEQUENCE LISTING

This application contains a Sequence Listing electronically submitted via EFS-Web to the United States Patent and Trademark Office as an XML file entitled “0110.000713US01.xml” having a size of 90,080 bytes and created on Sep. 11, 2025. The information contained in the Sequence Listing is incorporated by reference herein.

### SUMMARY

In one aspect, the present disclosure relates to a transgenic plant cell, the cell including a biocontainment system that includes: a coding region whose overexpression alters growth of the cell or impairs cell division; a transcription regulatory region operably linked upstream of the coding region, and including a mutation; and a polynucleotide that encodes a programmable transcription activator engineered to bind to the transcription regulatory region in the absence of the mutation after sexual reproduction, thereby overexpressing the coding region in the absence of the mutation, but does not initiate overexpression of the coding region when the transcription regulatory region includes the mutation.

In one or more embodiments, the transgenic plant cell is a germ cell. In one or more embodiments, the programmable transcription activator includes dCas9 fused to an activation domain. In one or more embodiments, the activation domain includes MoonTag or VP64. In one or more embodiments, the mutation includes a silent mutation. In one or more embodiments, the coding region encodes a developmental polypeptide, a cytoskeletal polypeptide, an ER-Golgi vesicle polypeptide, an mRNA processing polypeptide, an electron transport polypeptide, a nuclear trafficking polypeptide, a chromosome segregation polypeptide, a spindle pole duplication polypeptide, or an oxidative stress polypeptide.

In one or more embodiments, the coding region is the WUSCHEL gene or the LEC1 gene. In one or more embodiments, overexpression of the coding region is lethal to the cell. In one or more embodiments, the transgenic plant cell further includes a second biocontainment system including: a second coding region whose overexpression decreases growth of the cell or impairs cell division; a second transcription regulatory region operably linked upstream of the second coding region, and including a second mutation; a polynucleotide that encodes a second programmable transcription activator engineered to bind to the second transcription regulatory region in the absence of the second mutation, thereby overexpressing the second coding region in the absence of the second mutation, but does not initiate overexpression of the second coding region when the second transcription regulatory region includes the second mutation. In one or more embodiments, the second mutation includes a silent mutation.

In one or more embodiments, the altered cell growth includes a decrease in growth rate of a cell heterozygous for the biocontainment system compared to a suitable control. In one or more embodiments, the altered cell growth includes an increase in growth rate of a cell heterozygous for the biocontainment system, the increase in growth rate decreasing fitness of the cell compared to a suitable control. In one or more embodiments, the suitable control includes a wild-type cell. In one or more embodiments, the suitable control includes a cell homozygous for the biocontainment system.

In another aspect, the present disclosure relates to a plant including a transgenic plant cell described herein. In one or more embodiments, the plant is an oil-producing plant, a vegetable, a fruit, or a grain.

In another aspect, the present disclosure relates to a method of limiting hybridization of a genetically-modified plant with a genetically dissimilar variant, the method including: providing a plant genetically modified to include the biocontainment system of the present disclosure, wherein a cross between the genetically-modified plant and the genetically dissimilar variant plant results in progeny that exhibit a phenotype that is distinct from the genetically-modified plant. In one or more embodiments, the genetically dissimilar variant includes a wild-type plant. In one or more embodiments, the genetically dissimilar variant includes a different genetic modification compared to the genetically-modified plant having the biocontainment system. In one or more embodiments, the phenotype exhibited by the progeny includes lethality.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

FIG. 1. A schematic diagram illustrating engineered genetic incompatibility (EGI). (A) The macromolecular components that constitute programmable transcription factors (above), and schematic illustration showing lethal gene expression from a wild-type but not a refactored promoter (below). (B) An illustration of hybrid lethality upon mating of wild-type parents (right cell) and EGI parents (left cell). Macromolecular components are labeled in (A). Dark DNA signifies WT promoter, and light DNA signifies refactored promoter. (C) A cross between a wild-type organism (one that contains the parental promoter sequence) and an organism engineered to carry a refactored promoter will result in overexpression of the parental allele.

FIG. 2. Designing and testing sgRNAs to activate the WUSCHEL gene in Arabidopsis thaliana. (A) A schematic diagram showing the position of the four single guide RNAs (sgRNAs) designed to bind upstream of the WUSCHEL gene. (B) The relative expression of the WUSCHEL gene in protoplasts transfected with the SunTag activator and the indicated sgRNAs. Gene expression is shown relative to the housekeeping gene PP2A. “NOG” indicates the no sgRNA control.

FIG. 3. Generating mutations in the WUSCHEL promoter. (A) A schematic diagram of the T-DNA of the binary vector pMZ483 (SEQ ID NO:20) constructed to generate mutations in the WUS promoter. The Cas9 gene is driven by the RPS5a promoter (AtRps5a). The WUS sgRNA1 is driven by the Arabidopsis U6 promoter (AtU6). The TagRFP, OLE-RFP, gene of the FAST marker is driven by the AtOle promoter. The right border (RB) and the left border (LB) of the T-DNA are represented by rhombi. (B) The sequences of the WUSCHEL promoter mutants generated for WUS sgRNA1 using Cas9, with fixed +1 bp. A or T insertions were detected across multiple T2 lines. The Cas9 transgene was segregated away prior to mutation analysis. The spacer and PAM regions of sgRNA1 are indicated as underlined and boxed respectively.

FIG. 4. Generating prototype EGI in Arabidopsis. (A) A schematic diagram of the T-DNA of the binary vector pACM169 (SEQ ID NO: 19) constructed to express MoonTag together with the WUS sgRNA1. The genes encoding the different components of MoonTag are indicated. dCas9-24XGP41 and NbGP41-sfGFP-TAL are driven by the AtUBI10 promoter. The sgRNA1, indicated as gRNA1, is driven by the Arabidopsis U6 promoter (OsU6). The selectable marker NPTII's expression is driven by the 2× 35S promoter. The TagRFP marker is driven by the Arabidopsis OLE seed-specific promoter (OLE). The right border (RB) and the left border (LB) of the T-DNA are represented by grey triangles. (B) The prototype EGI Arabidopsis plant is homozygous for the mutation in the sgRNA1 in the WUS promoter and expresses the MoonTag activator along with the WUS sgRNA1. However, the MoonTag programmable transcription activator (PTA) will not activate the WUSCHEL gene because the mutation impairs the binding of the sgRNA1 to the promoter.

FIG. 5. The crosses between T1 EGI lines and wild-type Arabidopsis thaliana plants. Because T1 lines are heterozygous, only a fraction of the seeds will contain the T-DNA containing the MoonTag PTA. (A) The heterozygous seeds. (B) The seedlings from RFP (−) seeds (left panel) were phenotypically normal whereas the seedlings grown from RFP (+) seeds showed different phenotypes (right panel). (C) A size comparison between seedlings from a T1 EGI line, EGI(1-15), and the RFP(−) and RFP(+) progeny product of the cross of the EGI line with wild-type plants. (D) An RFP(+) seedling showing an ectopic embryo coming from root tissue. (E) An RFP(+) seedling displaying browning of the cotyledons.

FIG. 6. The phenotypes of RFP (+) plants grown from seeds produced by the cross between T1 EGI lines and wild-type Arabidopsis plants. (A) The RFP(+) positive seedlings resulted in plants with smaller size when transferred to soil. (B) The RFP(+) positive seedlings showed defects in flowers and silique formation.

FIG. 7. Expressing the indicated genes in the parental lines and the progeny of the cross between T1 EGI lines and wild-type Arabidopsis plants. (A) The results of a quantitative reverse transcriptase polymerase chain reaction (qRT-PCR) analysis of WUSCHEL expression in the indicated lines. (B) Expression of the nanobody component of MoonTag, NbGP41-sfGFP-TAL-GB1, was measured by qRT-PCR. Gene expression was normalized to that of Arabidopsis PP2A gene.

FIG. 8. The crosses between T2 EGI homozygous lines and wild-type Arabidopsis plants. (A) The bottom plates show the growth of the seedlings produced by the crosses between the wild-type and the indicated EGI lines. The top plates show the growth of seedlings corresponding to parental lines, the EGI 6-8 line and wild-type. The percentage of seedlings showing yellowing phenotypes in each plate is shown below each picture. The inset shows a magnification of the area indicated with the box. (B) WUSCHEL gene expression was measured by qRT-PCR in the crosses and the parental lines. Each point represents the mean expression from RNA isolated from 4-5 seedlings. The gray points in WTx3-8 indicate RNA isolated from seedlings showing the yellowing phenotype. Gene expression was normalized to that of Arabidopsis PP2A gene.

FIG. 9. The phenotypes of soil grown plants from the crosses between homozygous T2 EGI lines and Col-O wild-type plants. (A) the hybrid seedlings and parental lines from crosses with the EGI 5-15 line. (B) the hybrid seedlings and parental lines from crosses with the EGI 3-8 line. The seeds of the crosses and the indicated lines were planted directly on soil and grown for 23 days under long-day conditions. Germination percentages for each line are shown on the bottom.

FIG. 10. Plasmid maps of plasmids used herein.

FIG. 11. Plasmid maps of plasmids used herein.

FIG. 12. Plasmid maps of plasmids used herein.

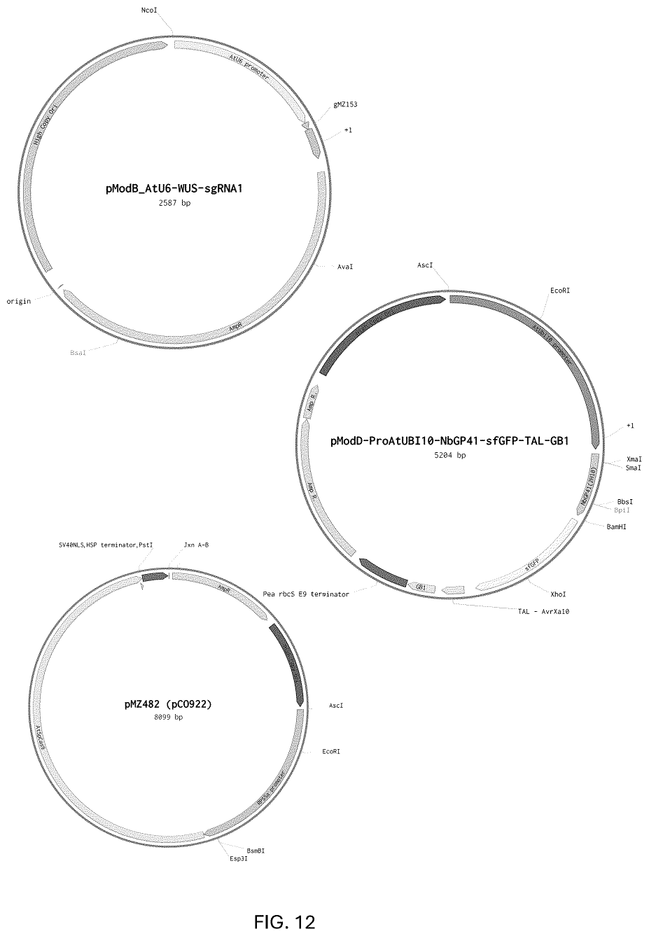

FIG. 13. A schematic diagram showing a general overview of synthetic incompatibility. (A) Silent mutations are introduced upstream of expression-sensitive coding regions. (B) A transcriptional activator is engineered to bind to the parental wild-type sequence. (C) A cross between a wild-type organism (one that contains the parental promoter sequence) and an organism engineered as shown in (A) and (B) will result in overexpression of the parental allele.

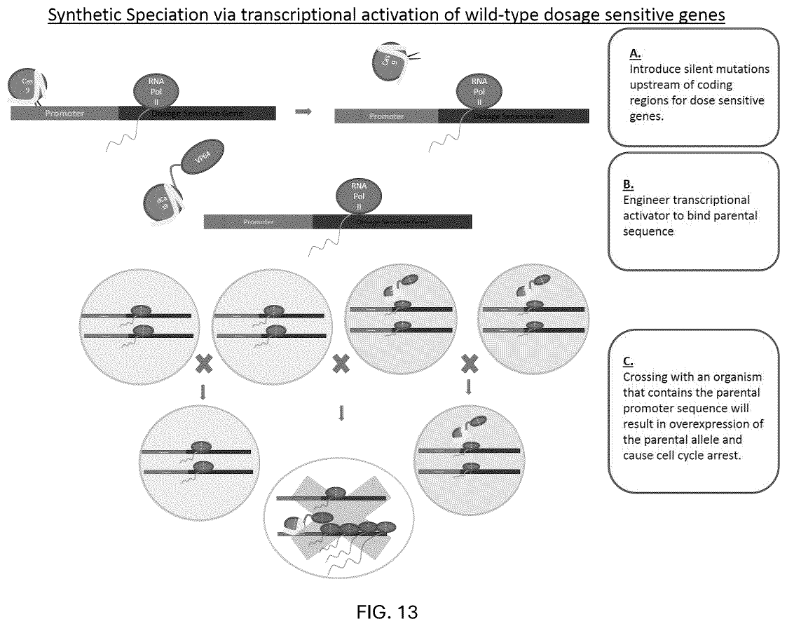

### DETAILED DESCRIPTION OF ILLUSTRATIVE EMBODIMENTS

This disclosure describes, in some aspects, a biocontainment system, cells and organisms that include such systems, and methods involving the construction and use of such systems. As used herein, the term “biocontainment system,” “biocontrol system,” and variations thereof refer to a genetic system that decreases the likelihood and/or extent to which a genetically-modified organism can sexually reproduce with a genetically dissimilar variant-whether wild-type or genetically modified in another way. In use, the system can decrease the likelihood and/or extent to which a genetic modification in, for example, a genetically-modified crop variety can spread into other variants. The system also can decrease the likelihood and/or extent to which a genetic modification can be diluted in a genetically-modified variety by the re-introduction of a wild-type genotype into a population of the genetically-modified variety.

Genetic recombination via sexual reproduction is a source of tremendous genetic variation and provides a mechanism for generating organisms with novel combinations of traits. Humans have exploited this process, often unknowingly, for domesticating plants and animals. Unwanted genetic recombination also takes place between domesticated and wild varieties of plants and animals. This is of particular concern in agricultural systems, particularly regarding seed production and organic (e.g., non-GMO) certification, because unwanted crosses may diminish the value of a crop. Farmers usually prevent unwanted crossing via spatial and temporal separation, which often requires co-ordination with neighboring farms. However, these methods are imperfect and can be labor intensive.

Recently, synthetic biology approaches have been used to genetically engineer plants to produce pharmaceutical and industrial compounds. These approaches emphasize the need for effective biocontainment strategies in order to prevent contamination of the food supply. Separating engineered plants from wild-type plants is often complicated and requires consideration of factors such as, for example, pollen size, duration of pollen viability, the presence of wild relatives, and typical meteorological conditions. Furthermore, for plants for which regulatory standards need to be met, these requirements are often yet more stringent. This creates a substantial burden for companies to use plant-based production systems.

Biocontainment approaches other than physical separation have been investigated, but each has at least one major drawback that prevents wide-spread adoption. For example, cleistogamy, in which flowers never open and therefore must self-pollinate is not applicable to all species. Maternal inheritance of transgenes by plastid engineering is likewise not applicable to all species, either because of the lack of maternal plastid inheritance or pollen-mediated transfer of plastids. Excising a transgene from a pollen-expressed recombinase may be highly efficient, but control of recombinase activity can interfere with normal propagation. Total sterility requires asexual propagation and is not practical for many species. Using exogenous chemical inputs to regulate lethal genes requires changes to normal cultivation techniques and genome reprogramming to confer dependence on synthetic compounds. Furthermore, with the exceptions of cleistogamy and asexual propagation, these methods only prevent outward gene-flow. Unwanted flow of genes into genetically engineered plants, however, can result in biocontainment failure in subsequent generations.

This disclosure describes Engineered Genetic Incompatibility (EGI) that allows for engineered species-like barriers to gene flow between otherwise sexually compatible populations. This stops the unwanted gene flow between engineered crops and their domestic and wild relatives without imposing significant changes to normal cultivation practices. EGI involves the use of Programmable Transcription Activator (PTA) technology to engineer a genetic incompatibility between two subpopulations by creating a biological speciation event between two otherwise sexually compatible populations. To do this, genetic relatedness is leveraged to engineer a lethal hybrid mating event only when an engineered species crosses with a dissimilar species, such as a wild-type species. Notably, due to the numerous potential coding regions that may be targeted by a PTA and multiple PTAs available, the biocontainment systems described herein may be used to engineer multiple distinct synthetic species from a single wild-type species. For example, a wild-type plant may be engineered to produce a first synthetic species including a first PTA targeting a first coding region and a second synthetic species including a second PTA targeting a second coding region. A cross between the first synthetic species and a wild-type plant may result in inviable or unfit progeny. Similarly, a cross between the first synthetic species and the second synthetic species may also result in inviable or unfit progeny. In other words, multiple versions of the biocontainment system described herein may be used in parallel. In this way, multiple engineered plants derived from the same species may be grown adjacent to each other without crossing.

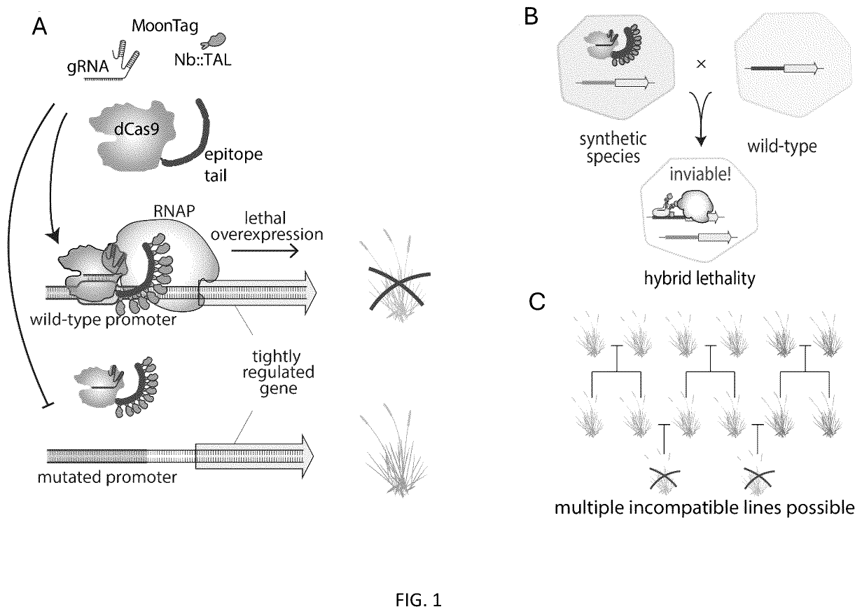

In one or more embodiments, PTAs are targeted to an endogenous gene promoter to drive overexpression of a target gene (FIG. 1A). Typically, overexpression of the gene of interest is detrimental or lethal to the progeny. In one or more embodiments, the gene of interest is a developmental gene. The EGI organism expresses PTAs which serve as sentinels for the single wild-type promoter sequence or numerous wild-type promoter sequences that are absent in the EGI organism. In one or more embodiments, an EGI-WT hybrid results in lethal gene activation when the PTA binds to the WT promoter sequence (FIG. 1). There are numerous potential target sites for PTAs. Thus, in one or more embodiments, multiple mutually incompatible sub-populations are constructed.

This disclosure describes a novel biocontainment approach in which a PTA monitors for the presence of a binding site upstream of an expression-sensitive coding region, e.g., any portion of the genome whose overexpression results in death or a severely deleterious phenotype. Thus, in one or more embodiments, overexpression of an expression-sensitive coding region reduces the reproductive compatibility of the organism in which the expression-sensitive coding region is overexpressed with dissimilar organisms. As it is used herein, a “dissimilar organism” is a genetically distinct organism, such as a wild-type organism or a differently engineered organism. In one or more embodiments, overexpression of a coding region makes an organism reproductively incompatible with a dissimilar organism. The upstream binding site is mutated in the engineered organism such that the PTA does not bind the upstream binding site (FIG. 1A). Typically, the PTA includes a nucleic acid-guided nucleic acid binding domain and an activation domain (AD). In one or more embodiments, the nucleic acid-guided nucleic acid binding domain is an RNA-guided DNA-binding domain, such as catalytically inactive (e.g., dead) Cas9 (dCas9). In one or more embodiments, the AD includes MoonTag, VP64, or a transcription-like activator (TAL).

After hybridizing with a wild-type organism, the PTA is able to bind to the non-mutated, wild-type upstream binding site contributed by the wild-type parent (FIG. 1B and FIG. 1C). In one or more embodiments, the binding causes overexpression of the expression-sensitive gene. In one or more embodiments, the overexpression is lethal. The engineered strain can effectively be considered a distinct species from the wild-type since it is no longer sexually compatible with the wild-type. This synthetic incompatibility approach does not require any changes in culture techniques or additional chemical inputs to maintain biocontainment. Furthermore, multiple orthogonal circuits can be introduced so that the same basic strategy can be used simultaneously in the same organismal background. Therefore, in one or more embodiments, more than one PTA is introduced, and more than one upstream binding site of an expression-sensitive coding region is mutated.

### Biocontainment Systems

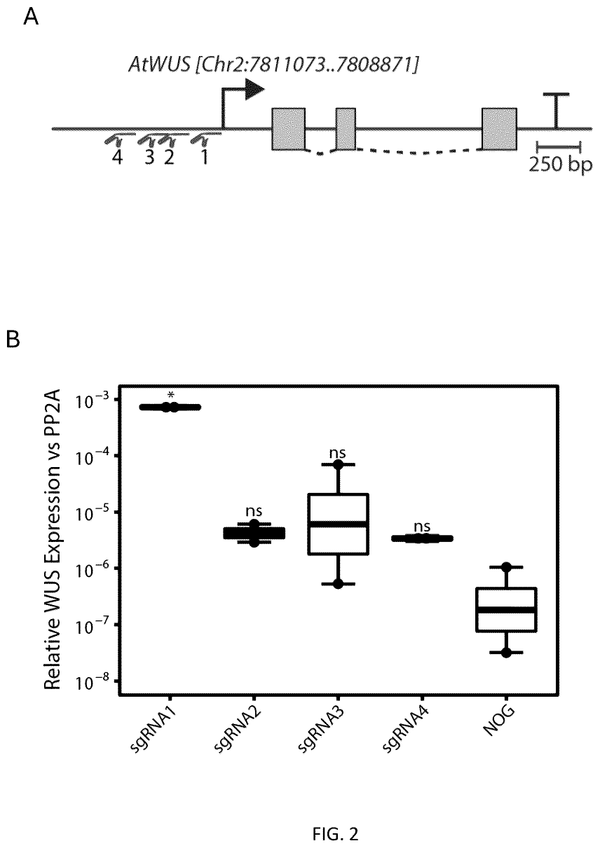

In one aspect, this disclosure relates to a biocontainment system so that the progeny of an organism that includes the system crossed with a wild-type organism exhibit reduced growth compared to a homozygous wild type organism. Generally, the system involves introducing a genetic barrier to sexual reproduction of a synthetically incompatible (SI) organism with a comparable wild-type organism of the same species. In one or more embodiments, the system involves the use of a PTA that can be used to lethally overexpress one or more endogenous expression-sensitive coding regions. Typically, lethality in the engineered synthetically incompatible strain is prevented by refactoring (e.g., mutating) the target locus, allowing the PTA to be expressed in the synthetically incompatible strain. This activator serves as a sentinel for undesired mating events, such as a cross between a synthetically incompatible organism and a wild-type organism. In one or more embodiments, hybridization between the synthetically incompatible strain and an organism containing the transcriptional activator's target sequence results in lethal expression of the expression-sensitive coding region (FIG. 2B).

In one or more embodiments, the biocontainment system is introduced into a plant species such as, for example, Arabidopsis thaliana. In one or more embodiments, the biocontainment system can be introduced into the cells of a multicellular organism. In one or more embodiments, the multicellular organism is a plant. Exemplary plants into which the biocontainment system may be introduced include, but are not limited to, a field crop (e.g., tobacco, corn, soybean, rice, etc.), a tree (e.g., poplar, rubber tree, etc.), and turfgrass (e.g. creeping bentgrass). In one or more embodiments, the plant is wheat, rye, sorghum, or sugar beet.

In one or more embodiments, the biocontainment system is present in a plant that can reproduce sexually. In one or more embodiments, the plant can reproduce sexually or asexually. In one or more embodiments, the plant cannot reproduce asexually. Plant sexual reproduction is mechanistically distinct from mammalian sexual reproduction. In one or more embodiments, the biocontainment system is present in a gametophyte of a plant. In one or more embodiments, the biocontainment system is present in a stamen of a plant, such as in the anther, the filament, or both the anther and the filament. In one or more embodiments, the biocontainment system is present in the carpel of a plant, such as the stigma, the style, the ovary, or a combination thereof. While described herein in the context of engineered male plants (e.g., pollen donors), it should be understood that the biocontainment systems described herein may be used in engineered female plants (e.g., pollen acceptors).

In one or more embodiments, the biocontainment system is present in a cell. In one or more embodiments, the cell is a gamete of a multicellular organism. In one or more embodiments, the cell is in a gametophyte of a plant. In one or more embodiments, the cell is a male gamete of a multicellular organism. Plant male gametes include, for example, pollen. In one or more embodiments, the cell is a female gamete of a multicellular organism. Plant female gametes include, for example, egg cells In one or more embodiments, the cell is a somatic cell of a multicellular organism. Types of plant somatic cells include, for example, parenchymal cells, collenchyma cells, sclerenchyma cells, xylem cells, or phloem cells. The plant cell may be part of any plant tissue, such as dermal, ground, meristematic, or vascular tissue.

Typically, the biocontainment system includes a genetically-modified cell that includes a coding region whose overexpression decreases growth of the organism, a transcription regulatory region operably linked upstream of the coding region and having a mutation, and a polynucleotide that encodes a programmable transcription activator. In one or more embodiments, the mutation is a silent mutation. In one or more embodiments, the mutation is a neutral mutation rather than a silent mutation. In other words, the mutation may not encode a biologically impactful change. In one or more embodiments, the programmable transcription activator is engineered to bind to the transcription regulatory region in the absence of the mutation. In one or more embodiments, the binding of the programmable transcription activator to the transcription regulatory region results in overexpression of the coding region in the absence of the mutation. Thus, in one or more embodiments where the organism is crossed with a wild type organism, the transcription activator initiates overexpression of the coding region and limits growth and/or viability of the organism in the absence of the mutation. In the presence of the mutation (e.g., when the organism is crossed with another organism having the same biocontainment system), the transcription activator does not initiate overexpression of the coding region and the progeny organisms remain viable.

As used herein, the term “overexpression” refers to a level of transcription of the coding region that is greater than that of a suitable wild-type control. The overexpression of the coding region that occurs when the organism is crossed with a wild-type organism results in altered growth of the organism so that one can identify organisms that are progeny of a cross with a wild type organism. Altered growth can include reduced growth compared to a comparable wild-type organism or can include increased growth compared to a wild-type organism that results is reduced fitness (e.g., a deformity that results in death). Altered growth may include an increased or decreased growth rate over a set period of time, increased or decreased total growth of the organism, or a combination thereof.

Overexpression can refer to ectopic expression, where genes are expressed in tissues where they are normally silent. Alternatively, or additionally, overexpression can refer to dysregulated expression, where the dynamic expression levels over time are perturbed such as, for example, a coding region that oscillates between an on-state and an off-state in wild-type that is constitutively in the on-state in the mutant.

In one or more embodiments, the result of cross between an organism having the biocontainment system and a wild-type organism can result in progeny that do not grow and/or are non-viable. In one or more embodiments, a cross between an organism having the biocontainment system and an organism not having the biocontainment system (e.g., a wild-type organism) results in no detectable progeny. In other cases, the result of cross between an organism having the biocontainment system and a wild-type organism can result in progeny that grow more slowly than organisms homozygous for the biocontainment system. Organisms that grow more slowly may be readily identifiable and may be culled from the population. Additionally or alternatively, organisms that grow more slowly may naturally be outcompeted by more fit organisms, effectively reducing or eliminating their presence in the growing space (e.g., field). In still other embodiments, the result of cross between an organism having the biocontainment system and a wild-type organism can result in progeny that grow more rapidly than organisms homozygous for the biocontainment system. Organisms that grow more rapidly may exhibit reduced fitness compared to the organisms homozygous for the biocontainment system. In one or more embodiments, a cross between an organism having the biocontainment system and a wild-type organism results in progeny that turn into calluses and fail to develop.

As used herein, the terms “silent mutation” and “neutral mutation” are used interchangeably and refer to a mutation in the DNA of the organism that does not significantly alter the phenotype of the organism outside of its effects within the context of the biocontainment system.

### Programmable Activators

As used herein, the term “programmable transcription activator” refers to a transcription activator whose DNA binding specificity can be programmed. In the context of the biocontainment system described herein, the transcriptional activator is programmed to survey the genome of a cell for the wild-type transcription regulatory sequence that controls transcription of the target coding region. The PTA typically does not bind to a variant of the transcription regulatory sequence that includes the mutation. While described herein in the context of an exemplary embodiment in which the programmable transcription activator is dCas9 fused to a VP64 or TAL AD, the biocontainment system may include any suitable PTA components. Typically, the PTA includes a nucleic acid binding protein and an AD. In one or more embodiments, the nucleic acid binding protein is a nucleic acid-guided nucleic acid binding protein. In one or more embodiments, the nucleic acid-guided nucleic acid binding protein is an RNA-guided DNA binding protein. In one or more embodiments, the nucleic acid-guided nucleic acid binding protein is a DNA-guided DNA binding protein. In one or more embodiments, the nucleic acid-guided nucleic acid binding protein includes all or a portion of Cas9, dCas9, or CPF1 (e.g., nuclease inactive CPF1). Typically, when the nucleic acid binding protein is a nucleic acid-guided nucleic acid binding protein, the PTA includes a guide nucleic acid.

In one or more embodiments, the nucleic acid binding protein is not guided by a nucleic acid, such as a transcription activator-like effector (TALE), a meganuclease, or a zinc finger DNA binding domain. In embodiments wherein the nucleic acid binding protein is not guided by a nucleic acid, the PTA typically does not include a guide nucleic acid.

In one or more embodiments, the AD includes all or a portion of VP64, VP16, VPR, p65, Rta, EDLL, Gal4, TAD, SunTag, the TALE AD from AvrXa10, HSFA6B, DREB1 from Arabidopsis sp., DREB2 from Arabidopsis sp., DOF 1 from Zea mays, or MoonTag. In one or more embodiments, the AD includes the nanobody component of MoonTag, sometimes referred to as “NbGP41.”

In one or more embodiments, activation of an RNA guided transcriptional regulator (e.g., dCas9-VP64) may be boosted by including aptamers in the RNA sequence which allow for the recruitment of aptamer binding protein such as, for example, transcription factor-fusions such as MS2/MCP, PP7/PCP, or COM fused to VP64, VP16, VPR, p65, Rta, and EDLL, Gal4, TAD or any combination thereof.

### Coding Regions

The coding region targeted for overexpression can be any coding region whose overexpression is detrimental to growth of the organism. Typically, overexpression of the coding region is detrimental to a degree sufficient to allow for easy identification and/or management of a hybrid cross between an organism having the biocontainment system and a comparable wild-type. In one or more embodiments, overexpression of the coding region can result from a cross between an organism having the biocontainment system and a comparable wild-type being lethal—e.g., the progeny of the cross do not grow or are otherwise non-viable. In one or more embodiments, overexpression of the coding region confers sensitivity to a crop management practice, such as an herbicide.

In one or more embodiments, the coding region encodes a developmental gene. In one or more embodiments, the coding region encodes a transcription factor, a chloroplast polypeptide, a cytoskeletal polypeptide, an ER-Golgi vesicle polypeptide, an mRNA processing polypeptide, an electron transport polypeptide, a nuclear trafficking polypeptide, a chromosome segregation polypeptide, a spindle pole duplication polypeptide, an oxidative stress polypeptide, a cell-signaling polypeptide, a pro-apoptotic polypeptide, a hypersensitive response-regulating polypeptide, or a developmental morphogen polypeptide. In one or more embodiments, the coding region encodes WUS (AT2G17950), LEC1 (AT1G21970), AtCSP4 (AT2G21060), STM (AT1G62360), MYB21 (AT3G27810), RSZ33 (AT2G37340), or CPSF73-I (AT1G61010). In one or more embodiments, the coding region encodes a transcription factor such as WUS or LEC1.

Typically, the coding region encodes an amino acid sequence. However, the coding region may encode a biomolecule that does not include an amino acid sequence, such as a long noncoding RNA (lncRNA), a micro RNA (miRNA), ribosomal RNA (rRNA), or transfer RNA (tRNA).

In embodiments wherein the PTA includes a nucleic acid-guided nucleic acid binding domain, the PTA includes a guide nucleic acid, such as a guide RNA (gRNA). The guide nucleic acid may be configured to bind a region near to the coding region, such as upstream of the coding region. In one or more embodiments, the guide nucleic acid includes at least 15, at least 16, at least 17, at least 18, at least 19, or at least 20 nucleotides. In one or more embodiments, the guide nucleic acid includes 10 to 40 nucleotides, such as 15 to 30 nucleotides, or 18 to 24 nucleotides. The guide nucleic acid is typically complementary to a region adjacent to the coding region.

In one or more embodiments, binding of the PTA to the transcription regulatory region results in at least a 10-fold increase in the expression of the coding region relative to expression of the coding region in the absence of the PTA. In one or more embodiments, binding of the PTA to the transcription regulatory region results in at least a 20-fold increase in the expression of the coding region relative to expression of the coding region in the absence of the PTA. In one or more embodiments, binding of the PTA to the transcription regulatory region results in at least a 40-fold increase in the expression of the coding region relative to expression of the coding region in the absence of the PTA. In one or more embodiments, binding of the PTA to the transcription regulatory region results in at least a 100-fold increase in the expression of the coding region relative to expression of the coding region in the absence of the PTA. In one or more embodiments, binding of the PTA to the transcription regulatory region results in at least a 120-fold increase in the expression of the coding region relative to expression of the coding region in the absence of the PTA. In one or more embodiments, binding of the PTA to the transcription regulatory region results in at least a 140-fold increase in the expression of the coding region relative to expression of the coding region in the absence of the PTA. In one or more embodiments, binding of the PTA to the transcription regulatory region results in at least a 500-fold increase, a 600-fold increase, an 800-fold increase, or a 1000-fold increase in the expression of the coding region relative to expression of the coding region in the absence of the PTA.

In one or more embodiments, binding of the PTA to the transcription regulatory region results in ectopic expression of the coding region. As it is used herein, “overexpression” may refer to increased expression or ectopic expression. Ectopic expression may refer to expression of a coding region in a cell or tissue in which it is typically not expressed and/or expression of a coding region at a time during which it is typically not expressed. For example, while expression of a developmental gene early in embryonic development may not be detrimental to an organism, expression of the same gene may be harmful or lethal in a fully developed organism.

In one or more embodiments, an organism may be engineered to include a second biocontainment system involving the programmed overexpression of a second coding region in the absence of a second mutation in the transcriptional regulatory region of the second coding region. In one or more embodiments, the second mutation is a silent mutation. The second mutation may alternatively encode a change that is not biologically meaningful. The second biocontainment system can include a second programmable transcription activator. The second programmable transcription activator may be the same as the first programmable transcription activator in all respects other than the transcription regulatory sequence it is programmed to survey. In other cases, the second transcription activator may include different components that the programmable transcription activator of the first biocontainment system.

### Transgenic Plant Cells

In one aspect, the present disclosure relates to a transgenic plant cell. In one or more embodiments, the transgenic plant cell includes a biocontainment system. In one or more embodiments, the biocontainment system includes a coding region whose overexpression alters growth of the cell or impairs cell division, a transcription regulatory region operably linked upstream of the coding region, and including a mutation, and a polynucleotide that encodes a programmable transcription activator (PTA) engineered to bind to the transcription regulatory region in the absence of the mutation after sexual reproduction. In the presence of the mutation, the PTA does not bind to the transcription regulatory region in a way that results in overexpression of the coding region. In the absence of the mutation, binding of the PTA to the transcription regulatory region results in overexpression of the coding region.

In one aspect, the transgenic plant cell including a biocontainment system is homozygous for the mutation in the transcription regulatory region operably linked upstream of the coding region. In one or more embodiments, the PTA does not bind to the transcription regulatory region in a way that results in overexpression of the coding region in the transgenic plant cell including a biocontainment system and that is homozygous for the mutation in the transcription regulatory region. In one or more embodiments, the transgenic plant cell including a biocontainment system that is homozygous for the mutation in the transcription regulatory region does not have overexpression or ectopic expression of the coding region. In one or more embodiments, the transgenic plant cell including a biocontainment system that is homozygous for the mutation in the transcription regulatory region does not have altered cell growth or impaired cell division compared to a plant cell of the same species that does not includes the biocontainment system.

In one aspect, the transgenic plant cell including a biocontainment system is heterozygous for the mutation in the transcription regulatory region operably linked upstream of the coding region. In one or more embodiments, binding of the PTA to the transcription regulatory region in the transgenic plant cell including a biocontainment system and that is heterozygous for the mutation in the transcription regulatory region results in overexpression of the coding region. In one or more embodiments, binding of the PTA to the transcription regulatory region in the transgenic plant cell including a biocontainment system and that is heterozygous for the mutation in the transcription regulatory region results in ectopic expression of the coding region. In one or more embodiments, overexpression or ectopic expression of the coding region alters growth of the cell or impairs cell division in the transgenic plant cell including a biocontainment system and that is heterozygous for the mutation in the transcription regulatory region. In one or more embodiments, overexpression or ectopic expression of the coding region is lethal to the transgenic plant cell including a biocontainment system and that is heterozygous for the mutation in the transcription regulatory region.

In one aspect, the transgenic plant cell includes more than one biocontainment system. In one or more embodiments, the first biocontainment system includes a first coding region whose overexpression alters growth of the cell or impairs cell division, a first transcription regulatory region operably linked upstream of the first coding region, and including a first mutation, and a first polynucleotide that encodes a first PTA engineered to bind to the first transcription regulatory region in the absence of the first mutation after sexual reproduction; and at least one other coding region whose overexpression alters growth of the cell or impairs cell division, at least one other transcription regulatory region operably linked upstream of the at least one other coding region, and including at least one other mutation, and at least one other first polynucleotide that encodes at least one additional PTA engineered to bind to the at least one other transcription regulatory region in the absence of the at least one other mutation after sexual reproduction.

In one or more embodiments, the transgenic plant cell is a germ cell. In one or more embodiments, the transgenic plant cell is a somatic cell.

### Transgenic Plants

In one aspect, the present disclosure relates to a plant. In one or more embodiments, the plant includes one or more transgenic plant cells described herein that include the biocontainment system. A plant including one or more transgenic plant cells may be referred to as a “transgenic plant.”

In one or more embodiments, the transgenic plant includes one or more transgenic plant cells described herein that include the biocontainment system and that are homozygous for the mutation in the transcription regulatory region operably linked upstream of the coding region. In one or more embodiments, the transgenic plant that includes one or more transgenic plant cells described herein that are homozygous for the mutation in the transcription regulatory region is not phenotypically distinct from a wild-type plant of the same species.

In one or more embodiments, the transgenic plant includes one or more transgenic plant cells described herein that include the biocontainment system and that are heterozygous for the mutation in the transcription regulatory region operably linked upstream of the coding region. In one or more embodiments, the transgenic plant that includes one or more transgenic plant cells described herein that are heterozygous for the mutation in the transcription regulatory region is phenotypically distinct from a wild-type plant of the same species. In one or more embodiments, the transgenic plant that includes one or more transgenic plant cells described herein that are heterozygous for the mutation in the transcription regulatory region is unable to produce progeny. In one or more embodiments, the progeny of the transgenic plant that includes one or more transgenic plant cells described herein that are heterozygous for the mutation in the transcription regulatory region has a lower germination percentage than a wild-type plant of the same species. In one or more embodiments, the transgenic plant that includes one or more transgenic plant cells described herein that are heterozygous for the mutation in the transcription regulatory region exhibits a yellowing phenotype compared to a wild-type plant of the same species. In one or more embodiments, the transgenic plant that includes one or more transgenic plant cells described herein that are heterozygous for the mutation in the transcription regulatory region has a smaller size compared to a wild-type plant of the same species. In one or more embodiments, the transgenic plant that includes one or more transgenic plant cells described herein that are heterozygous for the mutation in the transcription regulatory region exhibits defects in flowers and silique formation compared to a wild-type plant of the same species. In one or more embodiments, the transgenic plant that includes one or more transgenic plant cells described herein that are heterozygous for the mutation in the transcription regulatory region exhibits ectopic embryos. In one or more embodiments, the transgenic plant that includes one or more transgenic plant cells described herein that are heterozygous for the mutation in the transcription regulatory region exhibits cotyledon browning compared to a wild-type plant of the same species. In one or more embodiments, the transgenic plant exhibits increased sensitivity to herbicide application. In one or more embodiments, the transgenic plant exhibits developmental defects resulting in browning and senescence of somatic tissues.

In one or more embodiments, the transgenic plant stops developing further after germination and dies. In one or more embodiments, the transgenic plant develops into calli masses.

In one or more embodiments, the transgenic plant can be, but is not limited to, an oil-producing plant, a vegetable, a fruit, or a grain. In one or more embodiments, the transgenic plant can be, but is not limited to, tobacco, tomato, corn, soy, wheat, rye, sorghum, sugar beet, Nicotiana benthiana, or Arabidopsis thaliana.

### Methods of Making Transgenic Plants

In one aspect, the present disclosure relates to a method of producing a transgenic plant cell. The terms “transgenic plant” and “engineered plant” are used interchangeably herein. In one or more embodiments, the method includes introducing a biocontainment system into a plant cell. In one or more embodiments, the method includes selecting a coding region in a plant cell, the overexpression of which alters growth of the cell or impairs cell division. In one or more embodiments, the method includes selecting a transcription regulatory region operably linked upstream of the coding region, and introducing a mutation into the transcription regulatory region. In one or more embodiment, the method includes introducing into a plant cell a polynucleotide that encodes a programmable transcription activator engineered to bind to the transcription regulatory region in the absence of the mutation after sexual reproduction, wherein binding results in overexpression or ectopic expression of the coding region in the absence of the mutation, but does not initiate overexpression or ectopic expression of the coding region when the transcription regulatory region includes the mutation.

A method of producing a transgenic plant cell may include contacting a plant cell with a nucleic acid encoding one or more components of a biocontainment system. In one or more embodiments, a method of producing a transgenic plant cell includes contacting the plant cell with a nucleic acid encoding a programmable transcription activator. In one or more embodiments, a metho of producing a transgenic plant cell includes contacting the plant cell with editing reagents to introduce a genomic mutation in the plant cell, such as a silent mutation.

Contacting a plant cell with one or more reagents may include, for example, transforming the plant cell, such as by contacting the plant cell with a virus or bacterium. In one or more embodiments, transforming the plant cell includes a floral dip. In one or more embodiments, transducing the plant cell includes contacting the cell with agrobacterium. Typically, the agrobacterium includes a nucleic acid encoding editing reagents, a PTA, or both. In one or more embodiments, a method of producing a transgenic plant cell includes inserting a region encoding the PTA into the genome of the plant cell. The region encoding the PTA may be randomly inserted into the genome or may be inserted at a predetermined locus. In one or more embodiments, the region encoding the PTA may be inserted into the genome using a transposon.

In one aspect, the present disclosure relates to a method of growing a transgenic plant. In one or more embodiments, the method includes producing one or more transgenic plant cells described herein that include the biocontainment system, wherein the one or more transgenic plant cells is a germ cell or a somatic cell of the plant, and allowing the resulting transgenic plant to grow. In one or more embodiments, the method includes crossing a transgenic plant that includes one or more transgenic plant cells with a wild-type plant of the same species, and allowing the resulting progeny to grow.

### Methods of Limiting Hybridization

In another aspect, the present disclosure relates to a method of limiting hybridization of a genetically-modified plant with a genetically dissimilar variant. In one or more embodiments, the method includes providing a plant genetically modified to include the biocontainment system of any preceding claim, wherein a cross between the genetically-modified plant and the genetically dissimilar variant plant results in progeny that exhibit a phenotype that is distinct from the genetically-modified plant.

The systems and methods described herein typically limit hybridization, or production of cells that are heterozygous for a biocontainment system, without significantly affecting homozygous variants. In one or more embodiments, the genetically dissimilar variant is a wild-type plant. In one or more embodiments, the dissimilar variant includes a different genetic modification compared to the genetically-modified plant having the biocontainment system.

In contrast to conventional methods of limiting hybridization, the methods of the present disclosure typically do not require that plants are physically or temporally separated. Thus, a method of limiting hybridization may include growing a plant including the biocontainment system near a plant not including the biocontainment system.

In one or more embodiments, the phenotype exhibited by the progeny includes lethality. The phenotype exhibited by the progeny may additionally or alternatively be any phenotype described herein, such as altered growth or reduced ability to produce progeny.

In one aspect, the present disclosure relates to a method of reducing genetic crosses between transgenic plants. In one or more embodiments, the method includes allowing one transgenic plant that includes one or more transgenic plant cells described herein that include one biocontainment system with another transgenic plant of the same species that includes one or more transgenic plant cells described herein that include a different biocontainment system, wherein the resulting progeny is unable to produce its own progeny, produces progeny that has a lower germination percentage than a wild-type plant of the same species, exhibits a yellowing phenotype compared to a wild-type plant of the same species, has a smaller size compared to a wild-type plant of the same species, exhibits defects in flowers and silique formation compared to a wild-type plant of the same species, exhibits ectopic embryos, exhibits cotyledon browning compared to a wild-type plant of the same species, or a combination thereof.

In another aspect, the present disclosure relates to a method of reducing genetic crosses between transgenic plants and wild-type plants. In one or more embodiments, the method includes allowing one transgenic plant that includes one or more transgenic plant cells described herein that include a biocontainment system with a wild-type plant of the same species, wherein the resulting progeny exhibits a phenotype that is distinct from the genetically modified plant. In one or more embodiments, the phenotype includes inability to produce progeny, producing progeny that has a lower germination percentage than a wild-type plant of the same species, exhibiting a yellowing phenotype compared to a wild-type plant of the same species, smaller size, defects in flowers and silique formation, forming ectopic embryos, exhibiting increased cotyledon browning, or a combination thereof.

In another aspect, the present disclosure relates to a method of preventing the spread or dilution of a transgenic plant trait within a population. In one or more embodiments, the method includes allowing one transgenic plant that includes one or more transgenic plant cells described herein that include a biocontainment system with a wild-type plant of the same species, wherein the resulting progeny exhibits a phenotype that is distinct from the genetically modified plant. In one or more embodiments, the phenotype includes inability to produce progeny, producing progeny that has a lower germination percentage than a wild-type plant of the same species, exhibiting a yellowing phenotype compared to a wild-type plant of the same species, smaller size, defects in flowers and silique formation, forming ectopic embryos, exhibiting increased cotyledon browning, or a combination thereof.

### EXEMPLARY EMBODIMENTS

Embodiment 1 is a transgenic plant cell, the cell including a biocontainment system that includes:
a coding region whose overexpression alters growth of the cell or impairs cell division;
a transcription regulatory region operably linked upstream of the coding region, and including a mutation; and
a polynucleotide that encodes a programmable transcription activator engineered to bind to the transcription regulatory region in the absence of the mutation after sexual reproduction, thereby overexpressing the coding region in the absence of the mutation, but does not initiate overexpression of the coding region when the transcription regulatory region includes the mutation.

Embodiment 2 is the transgenic plant cell of embodiment 1, wherein the cell is a germ cell.

Embodiment 3 is the transgenic plant cell of embodiment 1 or embodiment 2, wherein the programmable transcription activator includes dCas9 fused to an activation domain.

Embodiment 4 is the transgenic plant cell of embodiment 3, wherein the activation domain includes MoonTag or VP64.

Embodiment 5 is the transgenic plant cell of any preceding embodiment, wherein the mutation includes a silent mutation.

Embodiment 6 is the transgenic plant cell of any preceding embodiment, wherein the coding region encodes a developmental polypeptide, a cytoskeletal polypeptide, an ER-Golgi vesicle polypeptide, an mRNA processing polypeptide, an electron transport polypeptide, a nuclear trafficking polypeptide, a chromosome segregation polypeptide, a spindle pole duplication polypeptide, or an oxidative stress polypeptide.

Embodiment 7 is the transgenic plant cell of embodiment 6, wherein the coding region is the WUSCHEL gene or the LEC1 gene.

Embodiment 8 is the transgenic plant cell of any preceding embodiment, wherein overexpression of the coding region is lethal to the cell.

Embodiment 9 is the transgenic plant cell of any preceding embodiment, further including a second biocontainment system including:
a second coding region whose overexpression decreases growth of the cell or impairs cell division;
a second transcription regulatory region operably linked upstream of the second coding region, and including a second mutation;
a polynucleotide that encodes a second programmable transcription activator engineered to bind to the second transcription regulatory region in the absence of the second mutation, thereby overexpressing the second coding region in the absence of the second mutation, but does not initiate overexpression of the second coding region when the second transcription regulatory region includes the second mutation.

Embodiment 10 is the transgenic plant cell of embodiment 9, wherein second mutation includes a silent mutation.

Embodiment 11 is the transgenic plant cell of embodiment 9 or embodiment 10, wherein the second coding region encodes a developmental polypeptide, a cytoskeletal polypeptide, an ER-Golgi vesicle polypeptide, an mRNA processing polypeptide, an electron transport polypeptide, a nuclear trafficking polypeptide, a chromosome segregation polypeptide, a spindle pole duplication polypeptide, or an oxidative stress polypeptide.

Embodiment 12 is the transgenic plant cell of any preceding embodiment, wherein the altered cell growth includes a decrease in growth rate of a cell heterozygous for the biocontainment system compared to a suitable control.

Embodiment 13 is the transgenic plant cell of any preceding embodiment, wherein the altered cell growth includes an increase in growth rate of a cell heterozygous for the biocontainment system, the increase in growth rate decreasing fitness of the cell compared to a suitable control.

Embodiment 14 is the transgenic plant cell of embodiment 12 or embodiment 13, wherein the suitable control includes a wild-type cell.

Embodiment 15 is the transgenic plant cell of embodiment 12 or embodiment 13, wherein the suitable control includes a cell homozygous for the biocontainment system.

Embodiment 16 is a plant including the transgenic plant cell of any preceding embodiment.

Embodiment 17 is the plant of embodiment 16, wherein the plant is an oil-producing plant, a vegetable, a fruit, or a grain.

Embodiment 18 is a method of limiting hybridization of a genetically-modified plant with a genetically dissimilar variant, the method including:
providing a plant genetically modified to include the biocontainment system of any preceding embodiment, wherein a cross between the genetically-modified plant and the genetically dissimilar variant plant results in progeny that exhibit a phenotype that is distinct from the genetically-modified plant.

Embodiment 19 is the method of embodiment 18 wherein the genetically dissimilar variant includes a wild-type plant.

Embodiment 20 is the method of embodiment 18 wherein the genetically dissimilar variant includes a different genetic modification compared to the genetically-modified plant having the biocontainment system.

Embodiment 21 is the method of embodiment 18 wherein the phenotype exhibited by the progeny includes lethality.

### EXAMPLES

### Example 1—EGI Target Gene Selection

The utility of synthetic incompatibility was established in the higher plant Arabidopsis thaliana. The approach first involved identifying genes that could be sufficiently overexpressed by the programmable transcription factor dCas9-VP64 to cause a strong defect in growth. Candidate genes and corresponding promoter regions were identified as targets for lethal overexpression phenotypes.

WUSCHEL (WUS) and LEAFY COTYLEDON 1 (LEC1) are developmental genes that are known to have lethal overexpression phenotypes (Lotan et al. Cell 93, 7 (1998): 1195-205). These genes are involved in early embryo formation and development. Ectopic postembryonic expression of these gene products results in aberrant somatic embryogenesis and the formation of embryo-like structures. Seedlings overexpressing WUSCHEL turn into calluses (calli) or cease to develop further after germination and die (Zuo et al. Plant J. 30, 3 (2002): 349-59). WUSCHEL was selected as a target gene for the prototype EGI, given the striking phenotypes displayed by its overexpression.

Four sgRNAs were designed to bind unique sequences upstream of NGG protospacer adjacent motif (PAM) sites in an approximately 250-nucleotide window upstream of predicted transcriptional start sites of the WUSCHEL transcriptional start site (TSS) (FIG. 2A). The sequences of the sgRNAs are shown in Table 1.

PTAs were targeted by guide RNAs (gRNAs) to regions upstream of the 5′ end of each gene's TSS result in strong transcriptional activation (Casas-Mollano et al. Nucleic Acids Res. 51, 13 (2023):7083-7093). Multiple locations to target within a promoter were identified.

Targeting multiple sgRNAs to a promoter boosts transcriptional activation. Using a single sgRNA for activation and mutagenesis decreases the likelihood of generating a large core promoter deletion. Because small indel mutations within a promoter region do not occur in a transcription factor binding site, they can generally be tolerated without significantly compromising native gene expression. Thus, in some embodiments, a single gRNA (sgRNA) was chosen for activation of the EGI. sgRNAs targeting four sites within 250 nucleotides of the transcriptional start site of the Arabidopsis thaliana WUSCHEL gene were designed and tested for activation in a protoplast transient system. The SunTag activation system, which was demonstrated to be an efficient activator of gene expression in Arabidopsis thaliana, was used to test the strength of each WUSCHEL sgRNA in the protoplasts (Papikian et al. Nat Commun. 2019; 10(1):729). This system utilized the AD scaffolding method, in which multiple ADs can be recruited to a single programmable DNA binding domain. This was accomplished by fusing a multimeric epitope tail to dCas9 while the ADs were fused to scFv antibodies. Each epitope is recognized by an antibody, resulting in recruitment of multiple scFv antibodies to a single dCas9 molecule (Tanenbaum et al. Cell. 2014; 159(3):635-646). For the assays, a customized version of the SunTag system was developed, in which 24 copies of the GCN4 epitope were fused to dCas9.

Arabidopsis thaliana protoplasts were isolated from 3-week-old plants and transfected with plasmids encoding SunTag (dCas9-24×GCN4 and scFv-AD) along with the sgRNAs. After transfection, RNA was isolated from the protoplasts and used to measure the expression of the WUSCHEL gene. From the four sgRNAs tested, WUS-sgRNA1, located closest to the TSS, produced the strongest WUSCHEL activation, which was more than three orders of magnitude higher than the WUSCHEL activation of the other three sgRNAs targeting the same promoter (FIG. 2B). Thus, WUS-sgRNA1 was selected for generating promoter mutations and for the prototype EGI.

TABLE 1
Sequences of the WUS sgRNA designed.

| | | Target sequence | |
|---|---|---|---|
| | SEQ | (including PAM) | |
| sgRNA | ID NO | 5′-3′ sequence | Strand |
| WUS-sgRNA1 | 1 | TAAATGGGGACCCA | antisense |
| | | AAAAGAAGG | |
| WUS-sgRNA2 | 2 | ACCCTATTGAAAAA | antisense |
| | | TGTGCTTGG | |
| WUS-sgRNA3 | 3 | AATAAATTTTGCCA | antisense |
| | | AAGAAAAGG | |
| WUS-sgRNA4 | 4 | ATAAAATGTTATGT | antisense |
| | | TTAGGAAGG | |

### Example 2—Generating Silent Mutations in the WUSCHEL Promoter

Since it was determined that WUS-sgRNA1 showed the strongest WUSCHEL activation among the tested guides, silent mutations that would impair WUS-sgRNA1 binding to the WUSCHEL promoter were created.

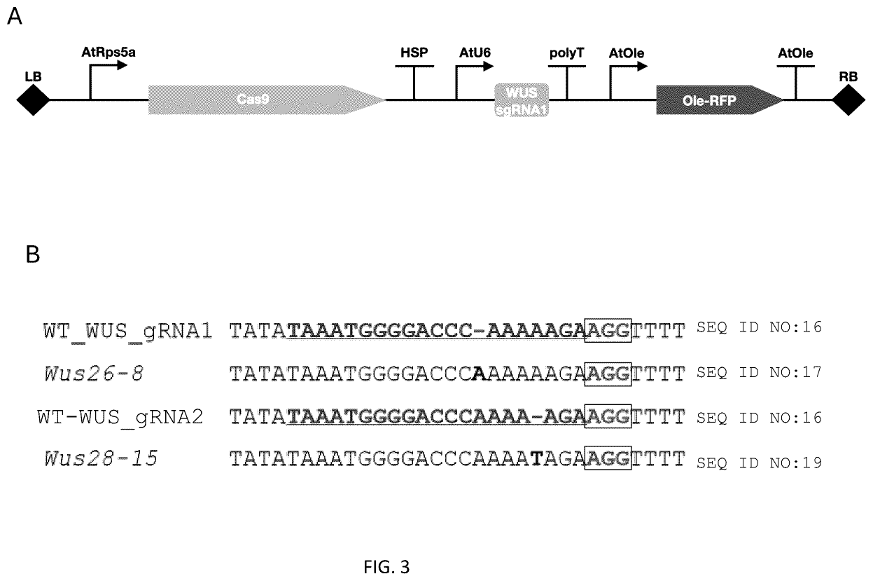

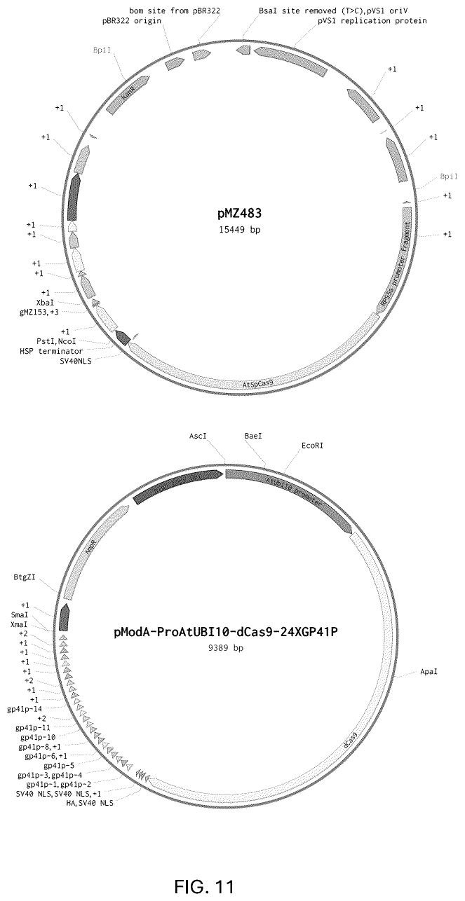

To generate a synthetically incompatible line with the desired silent mutation, a binary vector was designed to express a catalytically active version of Cas9 along with sgRNA1 in order to introduce a mutation in the sgRNA1 binding site (vector pMZ483, SEQ ID NO:20) (FIG. 3A and FIG. 11). The same vector pMZ483 also included a FAST marker gene for Cas9 selection (FIG. 3A and FIG. 11). T1 plants carrying the Cas9 T-DNA were identified using the FAST system by selecting for TagRFP positive seedlings (Shimada et al. Plant J. 2010; 61(3):519-528). Leaf tissue from these plants was collected for DNA extraction, followed by polymerase chain reaction (PCR) of the WUSCHEL promoter containing the sgRNA target site. Mutations were readily detected by Sanger sequencing in the T1 generation, with frequencies reaching up to 99% across 30 T1 lines that were screened. TagRFP negative seeds produced by the plants (T2 seeds), in which the Cas9 T-DNA was segregated out were identified. DNA isolation and PCR genotyping were carried out on T2 seedlings grown from these seeds to identify several plants containing fixed WUSCHEL promoter indel mutations (FIG. 3B).

Plants containing mutations such as A insertions or T insertions in the binding site of the sgRNA1 in the WUS promoter did not display any obvious morphological phenotypes or developmental aberrations, indicating that these mutations did not affect the proper functioning of the gene.

### Example 3—Engineered Genetically Incompatible Arabidopsis thaliana

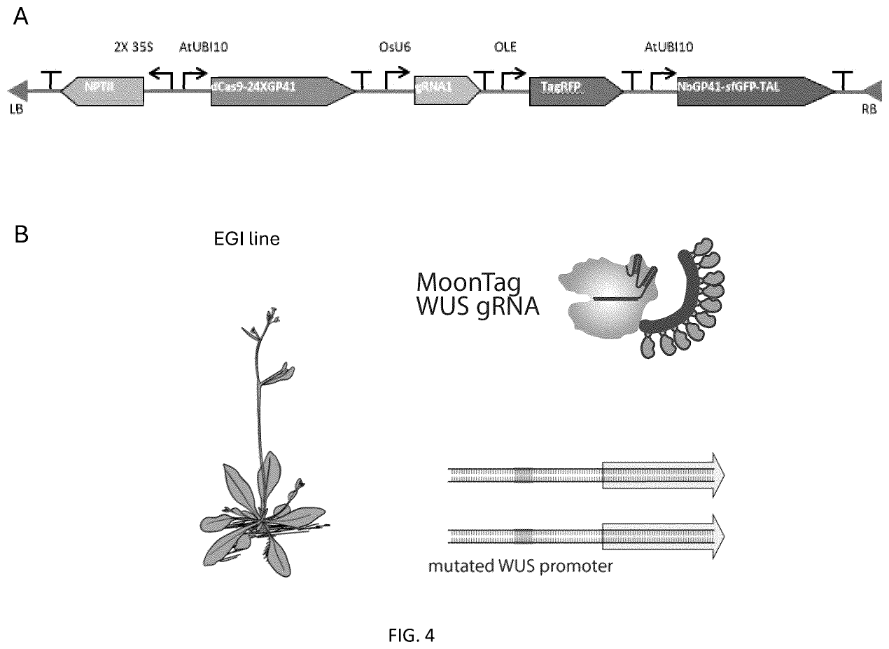

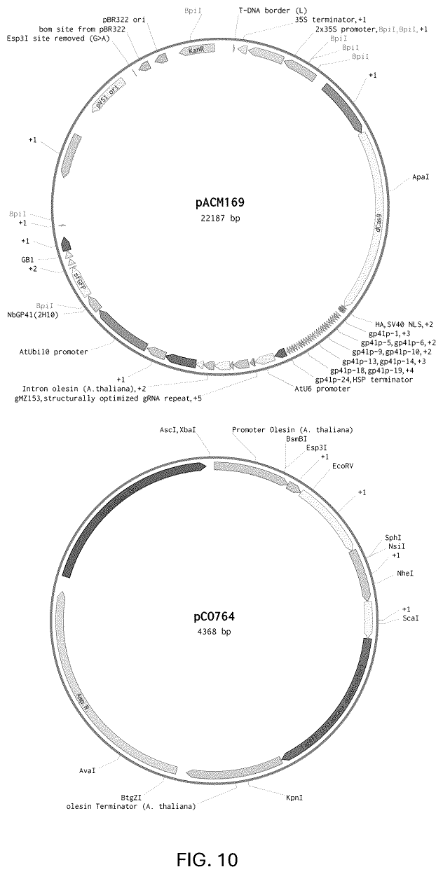

Two lines with neutral promoter mutations in the WUS promoter, one with an A insertion and another with a T insertion in the sgRNA1 biding site, were selected. The prototype EGI was created by transforming these lines with a T-DNA containing the PTA along with WUS sgRNA1 targeting the wild-type promoter sequence absent in the promoter mutant line (vector pACM169, SEQ ID NO:19) (FIG. 4A and FIG. 10). TagRFP seed-marker (FAST marker) was also included in order to follow the T-DNA during crosses. The PTA used here included the MoonTag activator, which efficiently activates endogenous genes and transgenes in several plant species, including in Arabidopsis thaliana (Casas-Mollano et al. CRISPR J. 2020; 3(5):350-364). A version of MoonTag in which the VP64 AD was replaced by the TAL AD was also used, since the TAL AD is capable of stronger activation (Zinselmeier et al. Plant Biotechnol J. 2024; 22(11):3202-3204, Zinselmeier et al. bioRxiv 2022).

The mutant plants were made to undergo floral dip transformation with Agrobacterium carrying the vector pACM169 (SEQ ID NO:19). TagRFP positive seeds were then recovered in both mutant lines. The TagRFP positive T1 seeds were germinated in soil and allowed to grow. The plants resulting from those seeds were not phenotypically different from parental mutant promoters and did not show developmental differences, indicating that the introduction of the MoonTag PTA with the WUS sgRNA1 did not cause any effect in the growth and development of the promoter mutants. This in turn indicates that the PTA was unable to activate the WUS gene in these lines.

### Example 4—Testing Prototype EGI Plant Lines

Next, the genetic compatibility between the EGI lines and plants containing the wild type promoter was examined. Floral dip plant transformation produced transgenes expressing the PTA to different levels. This resulted in lines capable of variable levels of activation of the target gene. However, because the mutation in the promoter of the EGI lines does not allow for activation of the WUS target, selection of lines capable of strong activation in the T1 progeny is not readily evident. Thus, in order to select for EGI lines conducive to strong activation of the WUSCHEL gene target, the T1 lines were crossed to the wild type, and those lines showing strong phenotypes and strong WUSCHEL activation were selected.

T1 lines having undergone floral dip transformation were heterozygous for the transgene insertion but homozygous for the promoter mutation. Therefore, only a fraction of the gametes coming from these plants contained the PTA, while all of them included the promoter mutation. For this reason, the crosses were carried out using the EGI lines as male (pollen donors) and the wild type as female plants. Thus, all the hybrid progeny contained one wild type copy and one mutated copy of the WUS promoter, while only some contained the PTA. Due to the addition of a TagRFP marker to the EGI construct, the progeny containing the PTA was identified by measuring the RFP fluorescence in the seed.

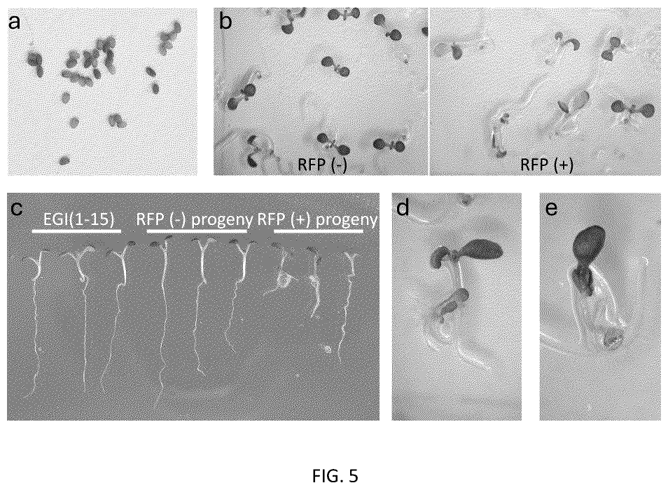

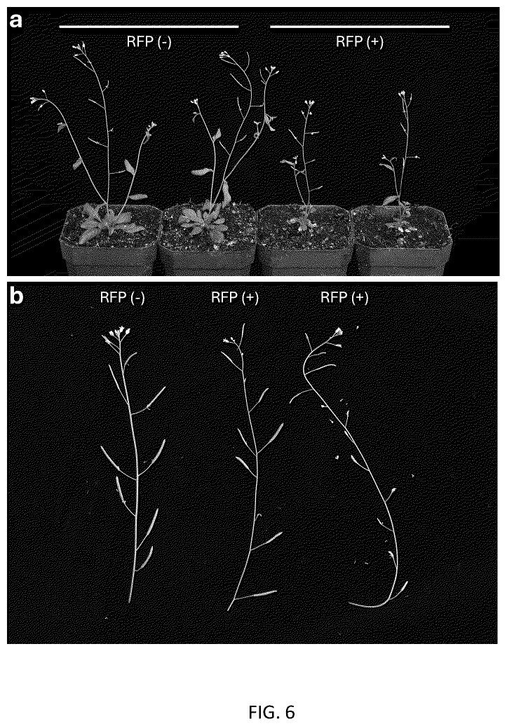

Only some of the seeds obtained after crossing EGI T1 lines to wild type plants expressed the TagRFP seed marker (FIG. 5A). The seeds resulting from the crosses were divided into RFP positive seeds (that contained the PTA) and RFP negative seeds (that did not contain the PTA) and were allowed to germinate in agar plates. Hybrid seedlings grown from RFP negative seeds were phenotypically normal, whereas hybrid seedlings grown from RFP positive seeds showed a range of phenotypes (FIG. 5B). RFP positive hybrid seedlings were smaller than those of the parental EGI line (FIG. 5C). In some cases, browning of the cotyledons was observed in the RFP positive hybrid seedlings, and a few of those seedlings developed ectopic embryos from the root tissue (FIG. 5D and FIG. 5E). An attempt to transfer the RFP positive hybrid seedlings to soil resulted in the death of some of those seedlings, while those that survived showed a range of developmental phenotypes, including smaller size and defects in flower and siliques (FIG. 6A and FIG. 6B). As shown in Table 2, the number of seedlings with observed phenotypes varied from cross to cross, depending on the T1 EGI lines (Table 2).

TABLE 2
Phenotypes observed in the progeny of the crosses
between T1 EGI lines and wild type plants

| | | Ectopic | | |
|---|---|---|---|---|
| Cross | RFP in seed | embryo | Phenotype * | Total seeds |
| EGI(5-15) × WT | + | 1 | 2 | 10 |
| | − | 0 | 0 | 10 |
| EGI(7-15) × WT | + | 4 | 5 | 11 |
| | − | 0 | 0 | 10 |
| EGI(8-15) × WT | + | 0 | 2 | 10 |
| | − | 0 | 0 | 12 |
| EGI(2-8) × WT | + | 1 | 4 | 10 |
| | − | 0 | 0 | 10 |
| EGI(5-8) × WT | + | 0 | 2 | 11 |
| | − | 0 | 0 | 10 |
| EGI(6-8) × WT | + | 0 | 4 | 11 |
| | − | 0 | 0 | 11 |

* Refers to seedlings either presenting ectopic embryos or with browning cotyledons

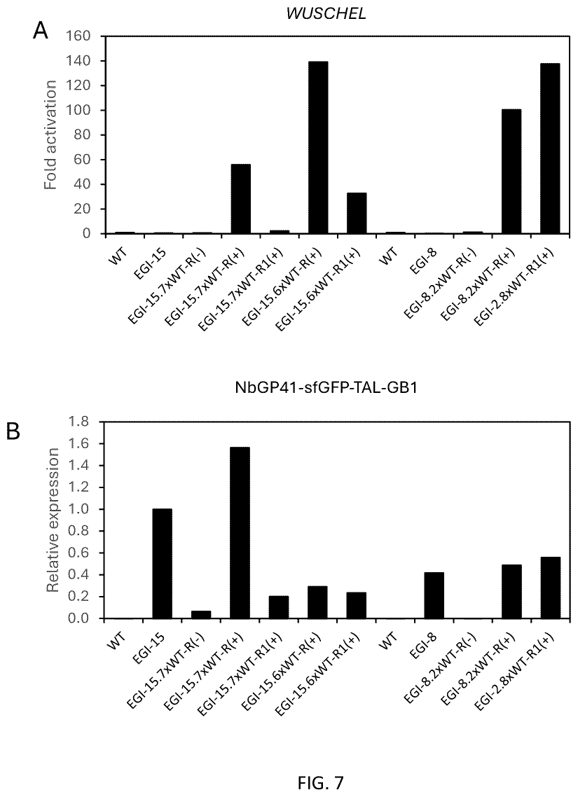

All the phenotypes observed in the RFP positive hybrid products resulting from the cross between the T1 EGI lines and the plants containing the wild type promoter were reminiscent of the defects observed from WUS overexpression, indicating that the combination of the PTA with the wild type WUS promoter resulted in gene activation. RT-qPCR analyses of the expression of WUSCHEL in the EGI lines, in the wild type plants, in the RFP-positive hybrid seedlings, and in the RFP-negative hybrid seedlings were conducted (FIG. 7A). RNA was isolated from 5 or 6 pooled 7-day old seedlings of the parental lines and the hybrids. WUSCHEL expression is restricted to meristematic regions and is absent from most differentiated tissues (Mayer et al. Cell. 1998; 95(6):805-815). Consequently, very low levels of WUSCHEL were detected in wild type seedlings. Similarly, very low levels of WUS were detected in the EGI lines generated with both promoter mutations, indicating that the silent mutations do not allow for the activation of the WUS gene in these lines (FIG. 7A). In contrast, different levels of WUS activation were observed in the RFP positive hybrid seedlings containing a combination of the wild type WUS promoter and the MoonTag PTA (FIG. 7A). No WUS activation was observed in RFP negative seedlings, which were not expected to contain the PTA (FIG. 7A). Expression of the nanobody component (NbGP41-sfGFP-TAL-GB1) of MoonTag was also analyzed in seedlings from the different lines and was found to be expressed, albeit to different levels, in both the EGI lines and the RFP-positive hybrids (FIG. 7B).

WUS expression data was used to identify the EGI lines that produced EGI target activation in the hybrids. From these selected lines, T2 seeds were grown and homozygous individuals were isolated. These homozygous plants grown from T2 seeds were used to carry out new crosses with wild type plants.

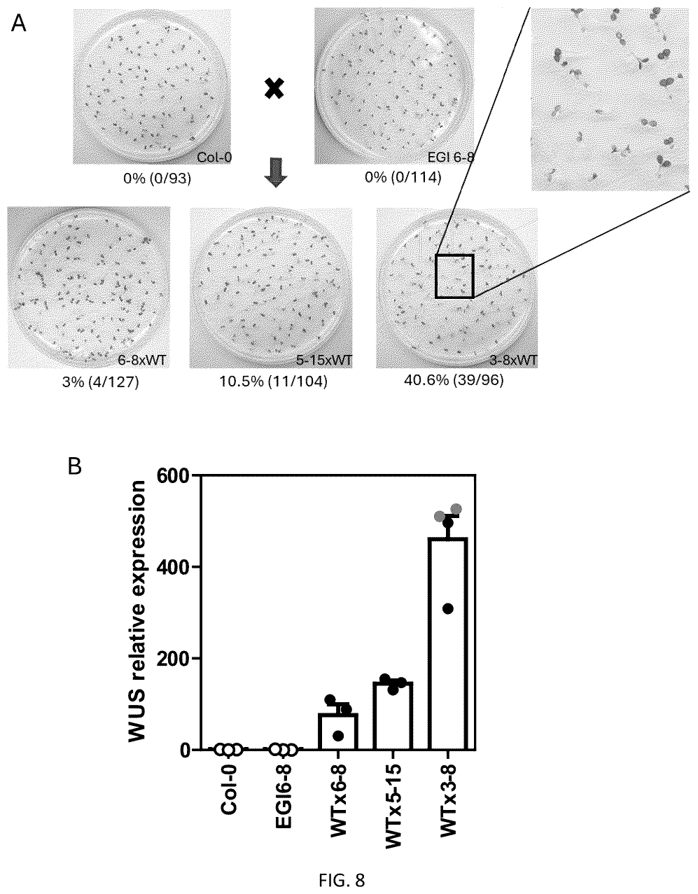

The majority of seeds obtained from the crosses between T2 homozygous EGI plants and the wild type were RFP positive, which was expected from the use of homozygous EGI lines in the crosses. RFP negative seeds were removed and the remaining hybrid seeds, as well as seeds from the parental line, were allowed to germinate in agar plates. The seedlings growing from the parental lines did not display any phenotypes (FIG. 8A). In contrast, a fraction of seedlings grown from the seeds obtained from the crosses showed yellowing similar to the brown phenotype that was observed in the crosses that were carried out with the T1 heterozygous lines (FIG. 8A). However, in contrast to earlier experiments, ectopic embryos were not observed.

RNA was isolated from these seedlings and RT-qPCR was used to measure the expression of WUSCHEL in the crosses and the parental lines (FIG. 8B). Similarly to what was observed in the crosses with the T1 EGI lines, only background levels of WUS were observed in the EGI lines and in the wild type parental plants. Conversely, hybrid seedlings showed increased expression of WUS relative to the parental lines (FIG. 8B). WUSCHEL activation in the progeny of the crosses with different EGI lines seemed to positively correlate with the fraction of seedlings showing yellowing phenotypes.

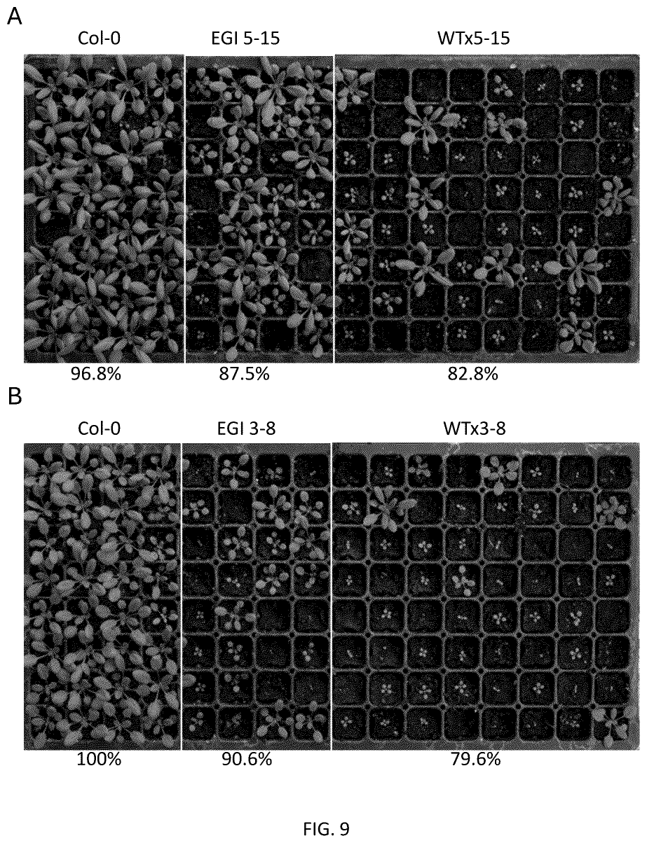

Seeds from the crosses from two T2 homozygous EGI lines showing the highest WUS activation (EGI 5-15 and EGI 3-8) were planted directly to soil and were grown under long-day conditions. Seeds coming from crosses between EGI lines and the wild type were severely delayed in germination and the resulting plants grew slower than the wild type (FIG. 9A and FIG. 9B). Seeds from both EGI lines were also delayed in germination, albeit to a lesser extent than their corresponding hybrid seedlings, and appeared to grow faster than them (FIG. 9A and FIG. 9B). Thus, even though there appeared to be a germination phenotype in the EGI lines, the hybrid seedlings appeared to germinate and grow slower than the EGI lines, which appeared to grow slower than the wild-type Col-0.

In the preceding description and following claims, the term “and/or” means one or all of the listed elements or a combination of any two or more of the listed elements; the terms “comprises,” “comprising,” and variations thereof are to be construed as open ended, e.g., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously. The particular examples, materials, amounts, and procedures are to be interpreted broadly in accordance with the scope and spirit of the invention as set forth herein.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

What is claimed is:

1. A transgenic plant cell, the cell comprising a biocontainment system that comprises:
a coding region whose overexpression alters growth of the cell or impairs cell division;
a transcription regulatory region operably linked upstream of the coding region, and comprising a mutation; and
a polynucleotide that encodes a programmable transcription activator engineered to bind to the transcription regulatory region in the absence of the mutation after sexual reproduction, thereby overexpressing the coding region in the absence of the mutation, but does not initiate overexpression of the coding region when the transcription regulatory region comprises the mutation.

2. The transgenic plant cell of claim 1, wherein the cell is a germ cell.

3. The transgenic plant cell of claim 1, wherein the programmable transcription activator comprises dCas9 fused to an activation domain.

4. The transgenic plant cell of claim 3, wherein the activation domain comprises MoonTag or VP64.

5. The transgenic plant cell of claim 1, wherein the mutation comprises a silent mutation.

6. The transgenic plant cell of claim 1, wherein the coding region encodes a developmental polypeptide, a cytoskeletal polypeptide, an ER-Golgi vesicle polypeptide, an mRNA processing polypeptide, an electron transport polypeptide, a nuclear trafficking polypeptide, a chromosome segregation polypeptide, a spindle pole duplication polypeptide, or an oxidative stress polypeptide.

7. The transgenic plant cell of claim 6, wherein the coding region is the WUSCHEL gene or the LEC1 gene.

8. The transgenic plant cell of claim 1, wherein overexpression of the coding region is lethal to the cell.

9. The transgenic plant cell of claim 1, further comprising a second biocontainment system comprising:
a second coding region whose overexpression decreases growth of the cell or impairs cell division;
a second transcription regulatory region operably linked upstream of the second coding region, and comprising a second mutation;
a polynucleotide that encodes a second programmable transcription activator engineered to bind to the second transcription regulatory region in the absence of the second mutation, thereby overexpressing the second coding region in the absence of the second mutation, but does not initiate overexpression of the second coding region when the second transcription regulatory region comprises the second mutation.

10. The transgenic plant cell of claim 9, wherein the second mutation comprises a silent mutation.

11. The transgenic plant cell of claim 9, wherein the second coding region encodes a developmental polypeptide, a cytoskeletal polypeptide, an ER-Golgi vesicle polypeptide, an mRNA processing polypeptide, an electron transport polypeptide, a nuclear trafficking polypeptide, a chromosome segregation polypeptide, a spindle pole duplication polypeptide, or an oxidative stress polypeptide.

12. The transgenic plant cell of claim 1, wherein the altered cell growth comprises a decrease in growth rate of a cell heterozygous for the biocontainment system compared to a suitable control.

13. The transgenic plant cell of claim 1, wherein the altered cell growth comprises an increase in growth rate of a cell heterozygous for the biocontainment system, the increase in growth rate decreasing fitness of the cell compared to a suitable control.

14. The transgenic plant cell of claim 12, wherein the suitable control comprises a wild-type cell or a cell homozygous for the biocontainment system.

15. A plant comprising the transgenic plant cell of claim 1.

16. The plant of claim 15, wherein the plant is an oil-producing plant, a vegetable, a fruit, or a grain.

17. A method of limiting hybridization of a genetically-modified plant with a genetically dissimilar variant, the method comprising:
providing a plant genetically modified to comprise the biocontainment system of any preceding claim, wherein a cross between the genetically-modified plant and the genetically dissimilar variant plant results in progeny that exhibit a phenotype that is distinct from the genetically-modified plant.

18. The method of claim 17 wherein the genetically dissimilar variant comprises a wild-type plant.

19. The method of claim 17 wherein the genetically dissimilar variant comprises a different genetic modification compared to the genetically-modified plant having the biocontainment system.

20. The method of claim 17 wherein the phenotype exhibited by the progeny comprises lethality.
